(19) United States
(12) Patent Application Publication
(10) Pub. No.: US 2020/0383785 A1
(43) Pub. Date: Dec. 10, 2020

(54) BIOENGINEERED ALLOGENEIC VALVE

(71) Applicant: VeriGraft AB, Gothenburg (SE)

(72) Inventors: Suchitra SUMITRAN-HOLGERSSON, Goteborg (SE); Antonio Rosales, Oslo (NO); Jonny Hisdal, Oslo (NO)

(73) Assignee: VeriGraft AB, Gothenburg (SE)

(21) Appl. No.: 16/892,763

(22) Filed: Jun. 4, 2020

Related U.S. Application Data
(62) Division of application No. 15/309,381, filed on Nov. 7, 2016, now abandoned.
(60) Provisional application No. 62/003,129, filed on May 27, 2014.

(51) Int. Cl. A61F 2/24; A61L 27/36; A61L 27/38; A61L 27/50; C12N 5/071

(52) U.S. Cl. CPC A61F 2/2475; A61L 27/3625; A61L 27/3886; A61L 27/507; A61L 27/3804; A61L 2430/40; A61L 27/3834; C12N 5/0691; A61L 27/367; A61L 2430/34; A61L 27/3826

(57) ABSTRACT

The present disclosure relates to methods for recellularization of valves in valve-bearing veins. This method is useful for producing an allogeneic venous valve, wherein a donor valve-bearing vein is decellularized and then recellularized using whole blood or bone marrow stem cells. The allogeneic valves produced by the methods disclosed herein are advantageous for implantation, transplantation, or grafting into patients with vascular diseases.

### RELATED APPLICATIONS

This application is a Divisional of U.S. application Ser. No. 15/309,381, which is the U.S. National Stage of PCT/EP2015/061736, filed May 27, 2015, which claims priority to, and the benefit of, U.S. provisional application No. 62/003,129, filed May 27, 2014, the disclosure of which is incorporated by reference in its entirety.

### FIELD OF DISCLOSURE

The present disclosure relates to methods for recellularization of valves in valve-bearing veins. The valves produced by the methods disclosed herein are advantageous for implantation, transplantation, or grafting into patients with vascular diseases.

### BACKGROUND

Venous valve is a membranous fold in a vein that prevents backward flow of blood passing through it. When a person is standing, blood must flow upward from the leg veins, against gravity, to reach the heart. The body has superficial veins, located in the fatty layer under the skin, and deep veins, located in the muscles and along the bones. Short veins, called connecting veins, link the superficial and deep veins. The deep veins play a significant role in propelling blood toward the heart. The one-way valves in deep veins prevent blood from flowing backward, and the muscles surrounding the deep veins compress them, helping force the blood toward the heart, just as squeezing a toothpaste tube ejects toothpaste. The powerful calf muscles are important, forcefully compressing the deep veins in the legs with every step. The deep veins carry 90% or more of the blood from the legs toward the heart.

One-way valves consist of two flaps (cusps or leaflets) with edges that meet. These valves help veins return blood to the heart. As blood moves toward the heart, it pushes the cusps open like a pair of one-way swinging doors (shown on the left). If gravity momentarily pulls the blood backward or if blood begins to back up in a vein, the cusps are immediately pushed closed, preventing backward flow (shown on the right).

Superficial veins have the same type of valves as deep veins, but they are not surrounded by muscle. Thus, blood in the superficial veins is not forced toward the heart by the squeezing action of muscles, and it flows more slowly than blood in the deep veins. Much of the blood that flows through the superficial veins is diverted into the deep veins through the many connecting veins between the deep and superficial veins. Valves in the connecting veins allow blood to flow from the superficial veins into the deep veins but not vice versa.

Chronic venous insufficiency (CVI) is a condition that occurs when the venous wall and/or valves in the leg veins are not working effectively, making it difficult for blood to return to the heart from the legs. CVI causes blood to “pool” or collect in these veins, and this pooling is called stasis. Veins return blood to the heart from all the body's organs. To reach the heart, the blood needs to flow upward from the veins in the legs. Calf muscles and the muscles in the feet need to contract with each step to squeeze the veins and push the blood upward. To keep the blood flowing up, and not back down, the veins contain one-way valves. Chronic venous insufficiency occurs when these valves become damaged, allowing the blood to leak backward. Valve damage may occur as the result of aging, extended sitting or standing or a combination of aging and reduced mobility. When the veins and valves are weakened to the point where it is difficult for the blood to flow up to the heart, blood pressure in the veins stays elevated for long periods of time, leading to CVI. An estimated 40 percent of people in the United States have CVI.

The conventional treatment of CVI with compression stockings combined with superficial surgery seems to improve venous hemodynamics; but only achieves 65% ulcer healing rate after 24 weeks and recurrence rate of 12%/year. Reconstructive deep venous surgical treatment of CVI and leg ulceration is invasive and, therefore, has been of limited use.

Blood moves through both arteries and veins because of the dynamic pressure derived from the pumping action of the heart. In a closed circulatory system, venous return must equal cardiac output. The majority of dynamic pressure is dissipated in the arterial circulation. The remaining energy is released in the venous system. Under normal circumstances, the pressure is 12 to 18 mm Hg at the venous end of the capillary and falls steadily toward arterial pressures of 4 to 7 mm Hg. When supine, gravitational pressures are neutralized and blood flows along this dynamic pressure gradient. Respiratory motion also strongly influences venous return in the supine position but has little effect when the extremity is dependent.

Hydrostatic pressure derives from the weight of the blood column below the right atrium. The density of blood and the acceleration of gravity determines hydrostatic pressure. Hydrostatic and gravitational pressures are expressed as a constant multiplier (0.77 mm Hg/cm) of the vertical distance in cm below the atrium. The pressure is highest in the upright (sitting or standing) but motionless individual. However, measured pressures also reflect external factors such as muscle contraction. Other external factors also alter flow through collapsible venous conduits. During inspiration, diaphragmatic contraction increases intra-abdominal and lower extremity venous pressure. Ascites and obesity produce similar increases in pressure even when supine.

Pressures in the dependent upper extremity reflect the vertical distance between the atrium and the first rib. Upper extremity veins that are held above the atria in an upright subject will collapse, as will extracranial veins of the head and neck. Pressures in venous structures above the atrium, therefore, do not fall below zero. Edema and reflux are uncommon in the upper extremity, even when venous valves are congenitally absent. Isolated central vein thrombosis often produces only a transient, proximal edema.

Venous return of blood from the dependent lower extremity to the heart is achieved by the ejection of blood by the lower extremity muscle pumps, assisted by competent venous valves. The valves function to divide the hydrostatic column of blood into segments and prevent retrograde venous flow. The greater number of valves in the infrapopliteal segment suggests their greater functional importance, but hydrostatic pressure can be significantly altered by the correction of femoral or popliteal vein incompetence. Perforating vein valves prevent deep to superficial flow, a concept consistent with the pressure/flow relationships of the calf pump. The perforating veins of the foot are an exception; bidirectional flow is considered normal.

Patients with chronic venous insufficiency (CVI) and leg ulceration constitute a serious medical and social problem with huge direct annual costs. The prevalence of venous leg ulcers is between 0.1 to 1.0%. The conventional treatment of CVI with compression stockings combined with superficial surgery seems to improve venous hemodynamics; but only achieves 65% ulcer healing rate after 24 weeks and recurrence rate of 12%/year.

In addition, the surgical technique is demanding and, therefore, has been of limited use in reconstructive deep venous surgery.

Thus, there remains need for improved methods for the treating patients inflicted with chronic venous insufficiency (CVI) and leg ulceration. The present disclosure provides alternative strategies to treat these disorders.

### SUMMARY OF THE DISCLOSURE

The present disclosure features, inter alia, materials and methods for decellularizing and recellularizing valves in valve-bearing veins.

The present disclosure provides successful preservation of valve function of tissue-engineered human valve-bearing vein segments under simulated in vivo physiological conditions. The present disclosure further provides the successful re-endothelialization of decellularized human vein segments and vein valves with a simple blood sample, from which endothelial cell monolayers are formed.

The present disclosure provides a method of recellularization of a valve in a vein, the method including introducing blood including progenitor cells for endothelial cells and progenitor cells for smooth muscle cells to the lumen of a decellularized vein, and culturing the cells in the lumen of the decellularized vein, thereby recellularizing the valve in the vein.

The present disclosure provides a method of recellularization of a valve in a valve-bearing vein, where the recellularized valve is a functional valve. In embodiments, the method includes harvesting a segment of a vein from a subject in need of the recellularized valve in a vein; decellularizing the valve-bearing vein; collecting blood from the subject, where the blood includes progenitor cells for endothelial cells and progenitor cells for smooth muscle cells; perfusing the decellularized valve-bearing vein with the collected blood; and culturing the cells in the decellularized valve-bearing vein, thereby recellularizing the decellularized valve in the vein.

In embodiments, the present disclosure includes a method of recellularizing a valve in a vein in which the recellularization restores competence and tolerance of reflux pressure of the recellularized valve.

The present disclosure provides method of treating chronic venous insufficiency (CVI), deep vein thrombosis (DVT), and/or leg ulceration in a subject (a recipient of a recellularized valve bearing vein) in need thereof by introducing a recellularized valve-bearing segment of a vein to the subject, where the valve is recellularized by a method including decellularizing a valve-bearing segment of an allogeneic vein; collecting blood from the subject, wherein the blood includes progenitor cells for endothelial cells and progenitor cells for smooth muscle cells; perfusing the decellularized valve-bearing with the collected blood; culturing the cells in the lumen of the decellularized valve-bearing vein; thereby recellularizing the decellularized valve of the vein; and grafting the recellularized valve-bearing vein to the subject, which treats CVI and/or leg ulceration in the subject.

In embodiments, blood is peripheral venous blood or whole blood. In embodiments, the peripheral venous blood or the whole blood is introduced to the decellularized vein by injection or perfusion. The method provides culturing the cells by perfusion of endothelial cell medium and smooth muscle cell medium. The perfusion of the endothelial cell medium and the smooth muscle cell medium may be in alternation. In embodiments, the recellularized valve is CD31 positive, vWF positive, smooth muscle actin positive, and has nuclei. In embodiments, the recellularized valve has mechanical properties of withstanding force at first peak at or above 0.8 N. In embodiments, the recellularized valve has a closure time of equal to or less than 0.5 seconds.

The present disclosure provides a method of recellularization of valves in veins by introducing one or more of: a population of endothelial cells, smooth muscle cells, progenitor cells for endothelial cells and progenitor cells for smooth muscle cells to the lumen of decellularized veins. The population of cells and/or progenitor cells is cultured with the decellularized veins, thereby recellularizing the valves in the veins. The recellularized valves of the present disclosure restore normal closure time (i.e., competence) and tolerate reflux pressure similar to normal healthy valve after being grafted in a subject in thereof. A feature of the present disclosure provides introducing a population of endothelial cells, smooth muscle cells, progenitor cells for endothelial cells and progenitor cells for smooth muscle cells to only the lumen of decellularized veins.

The present disclosure further provides a method of recellularization of a valve in a vein, wherein the recellularized valve is functional. The method of recellularization includes steps comprising: harvesting a valve-bearing segment of human vein; decellularizing the vein; collecting blood from a recipient of the recellularized valve; perfusing the decellularized vein with the collected blood for several hours (e.g., for about 2 hours, 3 hours, 4 hours, 5 hours, 6 hours, 7 hours, 8 hours, 9 hours, or about between 10-20 hours); perfusing the vein with endothelial cell media for more than one day; and perfusing the vein with smooth muscle media for more than one day; thereby recellularizing the decellularized valve of the vein. The recellularized valves using blood of the present disclosure restore normal closure time (i.e., competence) and tolerate reflux pressure similar to normal healthy valve after being grafted in a subject in need thereof.

The present disclosure provides a method of recellularization of a valve in a vein, wherein the recellularized valve is functional. The recellularization method includes steps: decellularizing a valve-bearing segment of vein harvested from a donor; perfusing with blood collected from the subject in need of treatment (i.e., in need of the valve recellularization in a vein) for several hours (e.g., for about 2 hours, 3 hours, 4 hours, 5 hours, 6 hours, 7 hours, 8 hours, 9 hours, or about between 10-20 hours); optionally perfusing the vein with endothelial cell media for more than one day; and perfusing the vein with smooth muscle media for more than one day; thereby recellularizing the decellularized valve of the vein, wherein the recellularization restores competence and tolerance of reflux pressure of the recellularized valve when grafted in a subject in need thereof.

In the method of valve recellularization, a population of cells, present or derived from peripheral venous blood or whole blood, are cultured with decellularized veins. The present disclosure provides that the population of cells is allogeneic. The present disclosure also provides that the population of cells is autologous. The population of cells, allogeneic and/or autologous, is collected from or is present in peripheral venous blood or whole blood. The present disclosure provides the peripheral venous blood or whole blood collected from an allogeneic donor. The peripheral venous blood or whole blood is autologous. The present disclosure provides culturing decellularized veins with allogeneic peripheral venous blood or allogeneic whole blood. The present disclosure provides culturing decellularized veins with allogeneic whole blood. The present disclosure also provides culturing decellularized veins with autologous peripheral venous blood or autologous whole blood.

The present disclosure provides decellularized veins. The veins are decellularized in a method involving repeated decellularization steps, i.e., decellularizing the veins more than once (defined as “cycles”). The vein decellularization involves between 2-16 cycles. For example, the decellularization method involves 14 cycles.

The valve-bearing veins of the present disclosure after decellularization in a method involving 2-16 decellularization cycles have reduced numbers of nuclei or are without nuclei (stains less or negative for nuclei in an immuohistochemical assay) compared to veins that are not decellularized. After 2-16 decellularization cycles, the veins are characterized as having reduced or no HLA class-I antigen (stains less or negative for HLA class-I antigen in an immuohistochemical assay), and/or have reduced or no HLA class-II antigens (stains less or negative for HLA class-II antigen in an immuohistochemical assay). After 2-16 decellularization cycles, the veins are characterized as having decreased collagen and sulfated glycosaminoglycans (GAG), and increased elastin compared to veins that are not decellularized. The decellularization decreases DNA in decellularized veins compared to veins that are not decellularized.

The present disclosure provides valve recellularization in decellularized valve-bearing veins, in which the recellularized valves and veins have nuclei and are CD31 positive. The veins with recellularized valves also are positive for smooth muscle actin, and have spindle shaped smooth muscle cells in the tunica adventitia of the vein. The recellularized valves and veins and are also positive for endothelial marker vWF.

The present disclosure provides recellularizing valves in decellularized veins, by a method that achieves functional valves. The functional valves have normal closure time (i.e., competent) and tolerate normal level of reflux pressure. Normal closure time of a valve with normal function, also referred to herein as competency, is less than or equal to 0.5 seconds. Valves with normal function tolerate the normal level of reflux pressure of 100 mm Hg, and reduced to a mean of about 18 mm Hg to about 25 mm Hg during walking 7 to 12 steps.

The present disclosure provides recellularized valves, which restores pressure of 12 to 18 mm Hg at the venous end of the capillary after grafting in a subject in need thereof. The recellularized valves restore normal hydrostatic and gravitational pressures in a subject in thereof, which are expressed as a constant multiplier (0.77 mm Hg/cm) of the vertical distance in cm below the atrium. The recellularized valves restore normal highest level of pressure in the upright (sitting or standing) but motionless subject after grafting, which is 100 mm Hg. The present disclosure also provides functional recellularized valves with a venous reflux pressure of about 100 mm Hg in an in vitro functional assay to mimic the flow through a vein in the deep venous system under the stress that occurs during walking and respiration.

The present disclosure provides recellularization of valves for achieving normal venous return of blood from the dependent lower extremity to the heart, by ejecting blood by the lower extremity muscle pumps, assisted by the competent recellularized venous valves. The recellularized valves of the present disclosure function to divide the hydrostatic column of blood into segments and prevent retrograde venous flow. The recellularization of veins restores the greater number of valves in the infrapopliteal segment. The recellularization of valves ameliorates symptoms resulting from hydrostatic pressure alteration by correcting femoral or popliteal vein incompetence. The perforating vein valves prevent deep to superficial flow, and restores normal pressure/flow relationships of the calf pump.

The recellularized valves in the perforating veins of the foot restore bidirectional flow of blood through the veins.

The present disclosure further provides a method of treating or ameliorating symptoms of chronic venous insufficiency (CVI) and/or leg ulceration in a subject in need thereof, involving grafting recellularized valve-bearing segments of veins to the subject. The valve recellularization method includes one or more steps. The method includes one or more steps: harvesting valve-bearing segments of human allogeneic femoral veins; decellularizing the veins in the femoral veins in 2-16 cycles; collecting blood from the subject; perfusing the decellularized veins with an anticoagulant; perfusing the decellularized veins with the collected blood for several hours; draining the blood and rinsing the vein with a buffer; and perfusing the veins first with endothelial cell media for more than one day, and then perfusing the veins with smooth muscle media for more than one day; and thereby recellularizing the decellularized valves; wherein the recellularized valve upon grafting to the subject treats or ameliorates CVI and/or leg ulceration.

The present disclosure provides a recellularised valve-bearing segment of blood vessel for use in treating or ameliorating symptoms of chronic venous insufficiency (CVI) and/or leg ulceration in a subject in need thereof. The recellularised segment of blood vessel is obtained by a method including one or more of the following steps: harvesting valve-bearing segments of human allogeneic femoral veins; decellularizing the veins in the femoral veins in 2-16 cycles; collecting blood from the subject; perfusing the decellularized veins with an anticoagulant; perfusing the decellularized veins with the collected blood for several hours; draining the blood and rinsing the vein with a buffer; and perfusing the veins first with endothelial cell media for more than one day, and then perfusing the veins with smooth muscle media for more than one day; and thereby recellularizing the decellularized valves; wherein the recellularized valve upon grafting to the subject treats or ameliorates CVI and/or leg ulceration; preferably wherein the recellularised segment of blood vessel is obtained by a method including all of the above-mentioned steps.

The present disclosure provides a method performed on a valve, which is a segment of a vein harvested or obtained from a body/donor, and blood collected from the subject to be treated, wherein the donor and the subject to be treated are not the same individual.

The valve decellularization steps of the present disclosure comprise treating the valve-bearing veins in a suitable detergents/organophosphate compounds including but are not limited to TRITON® and Tri-n-butyl phosphate and optionally additionally contain DNAase.

The decellularized valves are recellularized by perfusing the veins in endothelial cell media for between 2-6 days, followed by perfusing the veins in smooth muscle media for between 2-6 days. In one aspect, the culturing of the decellularized veins is in vitro.

The recellularized valves are CD31 positive. The recellularized valves can also be smooth muscle actin positive, vWF positive, and/or be characterized by the presence of spindle-shaped smooth muscle cells. The recellularized valves have mechanical properties of force at first peak above 0.8 N.

The present disclosure provides methods of treating recurrent leg cancer, which is due to deep venous reflux and/or venous hypertension, with recellularized valve-bearing veins. The present disclosure provides use of recellularized valve-bearing veins in treating recurrent leg cancer, which is due to deep venous reflux and/or venous hypertension.

The present disclosure features valves produced by any one of the recellularization methods described herein, wherein the recellularized valves are CD31 positive. The recellularized valves can also be smooth muscle actin positive, vWF positive, and/or be characterized by the presence of spindle-shaped smooth muscle cells. The recellularized valves have mechanical properties of force at first peak above 0.8 N.

The present disclosure also features the use of valves produced by any one of the methods described herein for implantation or grafting, wherein the recellularized valves are CD31 positive. The recellularized valves can also be smooth muscle actin positive, vWF positive, and/or be characterized by the presence of spindle-shaped smooth muscle cells. The recellularized valves have mechanical properties of force at first peak above 0.8 N.

The present disclosure provides recellularization of valves, which upon grafting in a subject in need thereof, treats and/or ameliorates the symptoms of incompetent valves in the thigh. In one aspect, the treatment and/or amelioration of the symptoms is achieved by restoring the normal working relationship between muscle pumps and the venous valves. The muscular pumps of the lower limb include those of the foot, calf, and thigh. Among these, the calf pump is the most important as it is most efficient, has the largest capacitance and generates the highest pressures (200 mm of mercury during muscular contraction). The normal limb has a calf volume ranging from 1500 to 3000 cc, a venous volume of 100 to 150 cc, and ejects over 40% to 60% of the venous volume with a single contraction.

During contraction, the gastrocnemius and soleus muscles drive blood into the large capacity popliteal and femoral veins. The recellularized valves of the present disclosure prevent retrograde flow (reflux) during subsequent relaxation, generating negative pressure and drawing blood from the superficial to the deep system through competent perforating veins. The recellularized valves incrementally lower venous pressure until arterial inflow equals venous outflow. When exercise ceases in a subject, the veins with recellularized valves slowly fill the capillary bed, causing a slow return to the resting venous pressure.

Although the thigh veins are surrounded by muscle, the contribution of thigh muscle contraction to venous return in minimal compared with the calf muscle pump. The planter venous plexus is compressed during ambulation and this pumping action is thought to prime the calf pump. Various leg pumps work together with competent valve function to return venous blood from the distal to proximal extremity.

Unless otherwise defined, all technical and scientific terms used herein have the same meaning as commonly understood by one of ordinary skill in the art to which this invention belongs. Although methods and materials similar or equivalent to those described herein can be used in the practice or testing of the present disclosure, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of the present disclosure are set forth in the accompanying drawings and the description below. Other features, objects, and advantages of the disclosure will be apparent from the drawings and detailed description, and from the claims.

### BRIEF DESCRIPTION OF THE DRAWINGS

FIG. 1 shows a photograph of the set-up and an in vitro model for functional testing of veins. The system is circulated with room temperate saline containing ultrasound contrast for enhancement of the Doppler signals. A peristaltic pump (a) pumps the saline through the whole circuit, a mechanical valve (b) enables flow through the vein (c) during output from the pump. An ultrasound probe (d) is used for visualization of the vein (e) and evaluation of the flow through the vein valve. Reflux pressure at the valve site is adjusted by the height of the reservoir (f) above the vein. A mechanical valve enables control of the direction of the flow and allows testing valve function. Valve-bearing venous segment is mounted in the circuit and placed in a container filled with saline to facilitate visualization by ultrasound; Valve closure time assessment; Reflux pressure at the valve site is adjusted by the height of the reservoir above the vein;

FIGS. 2A-2D show gross morphology and microscopic views of the decellularized valve-containing vein segments. (FIG. 2A) Gross morphology of normal vein and (FIG. 2B) decellularized vein after 14 cycles. HE staining of decellularized (FIG. 2C) vein after 14 cycles showing preserved tissue architecture and absence of blue-black nuclei and (FIG. 2D) a normal vein showing presence of nuclei.

FIGS. 3A-3D show the extracellular matrix in decellularized veins. (FIG. 3A) Masson's Trichrome (MT) staining of normal vein showing presence of nuclei (black), cytoplasm (red/pink) and collagen (blue). (FIG. 3B) In the decellularized vein (DV), no nuclei were found, indicating a lack of endothelial and smooth muscle cells, but staining for collagen indicates that collagen was still present. (FIG. 3C) Histogram showing significant decrease in amount of collagen & GAGs respectively after 14 decellularization cycles (p values are 0.03 and 0.005). (FIG. 3D) Histogram showing significant decrease in amount of DNA after decellularization (p value 0.0001). In FIG. 3A-3B, scale bar=50 μm.

FIGS. 4A-4F show the characterization of recellularized valve containing vein segments. (FIG. 4A) and (FIG. 4B) Hemotoxylin and eosin (HE) stained microscopic images of a recellularized vein and valve at low and high magnification respectively. The pictures show presence of continuous cells at endothelial lining on both vein and valve (arrow). (FIG. 4C) HE and Masson's Trichrome (MT) staining of normal and recellularized valves reveals presence of nuclei in all views of the veins. (FIG. 4D) CD31 staining of a recellularized vein reveals continuous endothelial lining. (FIG. 4E) Staining of smooth muscle actin of valves shows the presence of smooth muscle cells in the valve. (FIG. 4F) Staining of alpha smooth muscle actin shows smooth muscle cells in media of a recellularized vein.

FIGS. 5A-5E show images of the bioengineered vein grafts with autologous whole peripheral blood. (FIG. 5A) Immunofluorescence staining of tissue-engineered vein. Magnification is 100×. (FIG. 5B) Immunofluorescence image of bioengineered valve stained with anti-CD31 antibodies for the presence of endothelial cells (green) in the lumen. Magnification is 200×. (FIG. 5C) Negative control for the anti-CD31 antibody staining. Magnification is 200×. Immunohistochemical staining of tissue-engineered veins with anti-smooth muscle actin shows clear presence of smooth muscle cells (arrows) in the tunica adventitia layer. (FIG. 5D) Photograph of gross recellularized veins showing valves. (FIG. 5E) Photograph of veins with functional valves. The valves shown are bloated, indicating retention of liquid when injected with solution via a syringe in freshly harvested, decellularized and recellularized veins.

FIGS. 6A-6C show the mechanical analyses of the recellularized valves. (FIG. 6A) Representative diagrams of the deformation behavior for normal and recellularized (RC) vein valves. The valves were gradually torn apart horizontally resulting in a series of peaks. (FIG. 6B) Force at first peak for each pair of valves from the same vein. Normal vein valves are square marked while RC valves are circle shaped. Green color marks functioning veins while red colored do not function. The blue line represents a cut off for how high force at first peak a vein need to have to function (0.8N). (FIG. 6C) Box-plots of median force of working valves indicating no significant differences between normal valves and recellularized valves.

FIGS. 7A-7F show a series of ultrasound images showing the phases of functional testing tissue-engineered valve containing veins. FIGS. 7A-7F show a 2-D longitudinal ultrasound projections of the vein segment. A closed valve (FIG. 7A), and the gradual opening of the valve during antegrade flow (FIGS. 7B-7D) closing of the valve with retrograde flow (FIG. 7E) and (FIG. 7F).

FIGS. 8A-8C show a functional testing tissue-engineered valve containing veins. The yellow Doppler scale at the bottom of FIGS. 8A-8C shows the valve closure time in seconds.

FIGS. 9A-9B show a series of pictures demonstrating the biomechanical test. (FIG. 9A) Picture of a vein after cutting longitudinally to perform tear test and extension of valve while testing with instron tester. (FIG. 9B) Picture of the instron tester performing tear test on cut vein.

FIGS. 10A-10G show immunohistochemical analysis of decellularized veins. (FIG. 10A) DAPI staining of normal vein showing several nuclei, but (FIG. 10B) decellularized vein did not show DAPI-stained nuclei. (FIG. 10C) Immunohistochemical staining of decellularized veins showing absence of HLA class I antigen expression and (FIG. 10D) absence of HLA class II antigen expression. (FIG. 10E) Normal vein staining positive for HLA class I (brown) but not (FIG. 10F) HLA class II. (FIG. 10G) Negative control. Magnification in (FIG. 10A) 50×, (FIG. 10B) 100×.

FIGS. 11A-11D show recellularized veins. (FIG. 11A) Gross morphology of normal appearing pinkish in color (FIG. 11A) and recellularized veins (FIG. 11B). DAPI staining showing abundant nuclei in normal vein (FIG. 11C) and vein recellularized (FIG. 11D) with peripheral whole blood. Magnification at 100×.

FIG. 12A is a sketch diagram of the deep veins in the human leg. FIG. 12B is a diagram of one way valves when open or closed in a vein.

### DETAILED DESCRIPTION OF THE DISCLOSURE

The details of the present disclosure have been set forth in the accompanying description below. Although any methods and materials similar or equivalent to those described herein can be used in the practice or testing of the present disclosure, the methods and materials are described herein. Other features, objects, and advantages of the disclosure will be apparent from the description and from the claims. In the specification and the appended claims, the singular forms include plural references unless the context clearly dictates otherwise.

For convenience, certain terms used in the specification, examples and claims are collected here. Unless otherwise defined, all technical and scientific terms used herein have the same meaning as commonly understood by one of ordinary skill in the art to which this disclosure pertains.

### Definitions

As used herein, “recellularization” refers to the process of introducing or delivering cells to a decellularized vein and culturing the cells such that the cells proliferate and/or differentiate eventually to regenerate a valve with architecture, cell organization, and bioactivity similar to that of normal valve-bearing vein.

As used herein, “decellularization” refers to the process of removing cells from valves. Effective decellularization is dictated by factors such as tissue density and organization, geometric and biologic properties desired for the end product, and the targeted clinical application. Decellularization of valves with preservation of the ECM integrity and bioactivity can be optimized by those skilled in the art, e.g., by choosing specific agents and techniques during processing. The agents for decellularization may depend on many factors including cellularity, density, lipid content, and thickness of the vein. While most cell removal agents and methods may alter ECM composition and cause some degree of ultrastructure disruption, minimization of these undesirable effects is desired.

As used herein, “treatment” is an approach for obtaining beneficial or desired results, including clinical results. For purposes of this disclosure, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and recovery (whether partial or total), whether detectable or undetectable. “Treatment” can also mean prolonging survival as compared to expected survival if not receiving treatment.

By “reduce” or other forms of the word, such as “reducing” or “reduction,” is meant lowering of an event or characteristic (e.g., CVI and/or leg ulceration). It is understood that this is typically in relation to some standard or expected value, in other words it is relative, but that it is not always necessary for the standard or relative value to be referred to.

Insofar as the methods of the present disclosure are directed to preventing a disease or disease state, it is understood that the term “prevent” does not require that the disease state, e.g., CVI and/or leg ulceration, be completely thwarted. The term “prevent” can encompass partial effects when the recellularized valves disclosed herein are administered/grafted/transplanted as a measure reduce symptoms or progression of a disease, e.g., CVI and/or leg ulceration. The effects can extend from partial effect to differing degrees of effects, including an end effect of the individual being declared to be cured or lacking any symptoms of CVI and/or leg ulceration. The term does not require that the disease state be completely avoided at all times.

As used herein, “palliating” a disease or disease state means that the extent and/or undesirable clinical manifestations of a disease state are lessened (or alleviated) and/or time course of the progression is slowed or lengthened, as compared to not treating the disease.

As used herein, “inhibiting,” “inhibition,” and its various noun and verbal permutations are used herein to describe the biological effects of the recellularized valves. It does not necessarily require 100% inhibition. Partial inhibition is encompassed within this definition. Any degree of inhibiting as compared to the relevant control (e.g., a degree observed when the compound is not used) would be encompassed in the definition.

The competence of a valve, as used herein, refers to valve closure time, or the time from flow reversal until cessation of flow. The valve closure time of a valve with normal function is equal to or less than 0.5 seconds. In embodiments, the recellularized valves of the present disclosure may have a valve closure time (or competence) of equal to or less than 0.5 seconds.

Venous reflux, as used herein, refers to the backward flow of blood against the direction of blood flow toward the heart. A valve with normal function is able to withstand, or tolerate, reflux pressure of about 100 mm Hg and reduced to a mean of about 18 mm Hg to about 25 mm Hg during walking 7 to 12 steps. A valve with normal function remains closed, thereby preventing reflux, against pressure of up to 100 mm Hg and reduced to a mean of about 18 mm Hg to about 25 mm Hg during walking 7 to 12 steps. In embodiments, the recellularized valve may demonstrate an ability to withstand 10%, 20%, 30%, 40%, 50%, 60%, 70%, 80%, 90%, 100% of the reflux pressure tolerated by normal valves, i.e., 100 mm Hg.

As used herein, “subject” means a human or animal (in the case of an animal, more typically a mammal). In one aspect, the subject is a human. In one aspect, the subject is a male. In one aspect, the subject is a female. As used herein, a “subject in need thereof” is a subject having a vascular disease or disorder associated with diseased or incompetent valves, or a subject having an increased risk of developing such a vascular disease or disorder that requires a vascular graft or transplant relative to the population at large.

For the purposes of promoting an understanding of the disclosure herein, reference made to features and specific language are used to describe the same. The terminology used herein is for the purpose of describing particular features only, and is not intended to limit the scope of the present disclosure. As used throughout this disclosure, the singular forms “a,” “an,” and “the” include plural reference unless the context clearly dictates otherwise. Thus, for example, a reference to “a valve” includes a plurality of such compositions, as well as a single composition. All percentages and ratios used herein, unless otherwise indicated, are by weight.

A weight percent of a component, unless specifically stated to the contrary, is based on the total weight of the formulation or composition in which the component is included. The term “about” refers to any minimal alteration in the concentration or amount of an agent that does not change the efficacy of the agent in preparation of a formulation and in treatment of a disease or disorder. The term “about” with respect to concentration range of the agents (e.g., therapeutic/active agents) of the current disclosure also refers to any variation of a stated amount or range which would be an effective amount or range.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Venous blood is deoxygenated blood, which travels from the peripheral vessels, through the venous system into the right atrium of the heart. Deoxygenated blood is then pumped by the right ventricle to the lungs via the pulmonary artery, which is divided in two branches, left and right to the left and right lungs respectively. Blood is oxygenated in the lungs and returns to the left atrium through the pulmonary veins.

Post-natal vasculogenesis is the formation of new veins in adults by circulating endothelial progenitor cells (EPCs); and angiogenesis is formation of new veins from pre-existing endothelial cells (Ribatti D et al., 2001). These two processes contribute in formation of vein branches and in pathogenic states like wound healing, ischaemia, fracture healing, tumor growth etc., (Laschke et al, 2011).

The present disclosure includes that population of cells used to recellularize the vein is allogeneic. “Allogeneic” as used herein refers to blood obtained from an individual of the same species as that from which the organ or tissue originated (i.e., related or unrelated individual).

As used herein, autologous means the donor and recipient are the same person. For example, patients scheduled for non-emergency surgery may be autologous donors by donating blood for themselves that will be stored until the surgery. An autologous graft is a graft (such as a graft of skin) that is provided for oneself. In embodiments, the method of recellularization of the present disclosure includes introducing blood or cells obtained from the recipient (i.e., the subject in need of treatment (i.e., “autologous”).

For the purposes of promoting an understanding of the disclosure herein, reference made to features and specific language are used to describe the same. The terminology used herein is for the purpose of describing particular features only, and is not intended to limit the scope of the present disclosure. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

Blood moves through both arteries and veins because of the dynamic pressure derived from the pumping action of the heart. In a closed circulatory system, venous return must equal cardiac output. The majority of dynamic pressure is dissipated in the arterial circulation. The remaining energy is released in the venous system. Under normal circumstances, the pressure is 12 to 18 mm Hg at the venous end of the capillary and falls steadily toward arterial pressures of 4 to 7 mm Hg. When supine, gravitational pressures are neutralized and blood flows along this dynamic pressure gradient. Respiratory motion also strongly influences venous return in the supine position but has little effect when the extremity is dependent.

Hydrostatic pressure derives from the weight of the blood column below the right atrium. The density of blood and the acceleration of gravity determines hydrostatic pressure. Hydrostatic and gravitational pressures are expressed as a constant multiplier (0.77 mm Hg/cm) of the vertical distance in cm below the atrium. The pressure is highest in the upright (sitting or standing) but motionless individual. However, measured pressures also reflect external factors such as muscle contraction. Other external factors also alter flow through collapsible venous conduits. During inspiration, diaphragmatic contraction increases intra-abdominal and lower extremity venous pressure. Ascites and obesity produce similar increases in pressure even when supine.

Pressures in the dependent upper extremity reflect the vertical distance between the atrium and the first rib. Upper extremity veins that are held above the atria in an upright subject will collapse, as will extracranial veins of the head and neck. Pressures in venous structures above the atrium, therefore, do not fall below zero. Edema and reflux are uncommon in the upper extremity, even when venous valves are congenitally absent. Isolated central vein thrombosis often produces only a transient, proximal edema.

Venous return of blood from the dependent lower extremity to the heart is achieved by the ejection of blood by the lower extremity muscle pumps, assisted by competent venous valves. The valves function to divide the hydrostatic column of blood into segments and prevent retrograde venous flow. The greater number of valves in the infrapopliteal segment suggests their greater functional importance, but hydrostatic pressure can be significantly altered by the correction of femoral or popliteal vein incompetence. Perforating vein valves prevent deep to superficial flow, a concept consistent with the pressure/flow relationships of the calf pump. The perforating veins of the foot are an exception; bidirectional flow is considered normal.

Reconstructive deep venous surgery such as valvuloplasty, auto-transplantation and neovalve construction has proved to be an option improving ulcer-healing rates and providing ulcer-free period, in patients where conventional treatment has failed. However, the durability of these procedures remains an issue. See Rosales A. et al., Venous valve reconstruction in patients with secondary chronic venous insufficiency, EUR. J. OF VASCULAR AND ENDOVASCULAR SURGERY, (2008), 36:466-72; see also Rosales A. et al., External venous valve plasty (EVVP) in patients with primary chronic venous insufficiency (PCVI), EUR. J. OF VASCULAR AND ENDOVASCULAR SURGERY, (2006), 32:570-576; and Maleti O. & Perrin M., Reconstructive surgery for deep vein reflux in the lower limbs: techniques, results and indications. EUR. J. OF VASCULAR AND ENDOVASCULAR SURGERY (2011), 41:837-48.

The present disclosure is based on the surprising discovery that venous valves suitable for surgical implantation can be successfully bioengineered from a deceased donor vein that was decellularized and later recellularized by autologous cells from the recipient of the graft. This approach can be considered for patients in need of bypass surgery or vascular vein shunts due to thrombosis, chronic deep vein incompetence, vein obstruction or venous reflux. Further, this technique obviates the need for life-long immunosuppression, and is a promising and safe clinical approach with great benefits and lower risks than previous vascular transplant solutions.

The present disclosure provides methods for decellularizing a vein. Methods for decellularization of veins encompass the removal of endogenous cells while preserving integrity of the extracellular matrix (ECM) are described herein. The process of decellularization as described herein utilizes sequential treatment of two or more different cellular disruption solutions, in several cycles. In a feature of the present disclosure, decellularization may be achieved when no nuclei remain, as detected by various methods known in the art. The vein may be from a donor. In the present disclosure, the veins for recellularization are obtained from a donor; the donor being deceased. The donor may be from a HLA or tissue-matched donor.

The present disclosure also provides methods for recellularization of the decellularized valves, comprising introducing a population of cells to the decellularized veins and culturing said population of cells on and in the decellularized veins. Methods described herein are useful for the expansion of the population of cells and differentiation of the population of cells to functional endothelial cells and smooth muscle cells to produce functional valves.

The present disclosure provides recellularizing valves in the venous system of the lower extremities. The venous system of the lower extremities includes the deep veins, which lie beneath the muscular fascia and drain the lower extremity muscles; the superficial veins, which are above the deep fascia and drain the cutaneous microcirculation; and the perforating veins that penetrate the muscular fascia and connect the superficial and deep veins. Communicating veins connect veins within the same compartment. The superficial, deep, and most perforating veins contain bicuspid valves that assure unidirectional flow in the normal venous system. The present disclosure provides recellularization of bicuspid valves and use of the recellularized bicuspid valves in restoring unidirectional flow of the venous system.

The present disclosure provides recellularization of valves in the great saphenous vein and the anterior and posterior accessory great saphenous veins of the superficial veins. The principle veins of the medial superficial system are the great saphenous vein and the anterior and posterior accessory great saphenous veins. The present disclosure provides that valves of the saphenous subcompartment and saphenous fascia are recellularized. The saphenous fascia covers the saphenous subcompartment and separates the great saphenous vein from other veins in the superficial compartment. The present disclosure provides that valves in the small saphenous vein (SSV) are recellularized. The SSV is the most important posterior superficial vein of the leg. It originates from the lateral side of the foot and drains into the popliteal vein, most commonly joining it just proximal to the knee crease. The present disclosure provides that valves in the intersaphenous vein are recellularized. Intersaphenous vein (previously termed the vein of Giacomini) connects the small and great saphenous veins.

The present disclosure provides recellularization of valves in the superficial femoral vein (a deep vein). The superficial femoral vein (a deep vein) (also known as the femoral vein), connects the popliteal vein to the common femoral vein. The present disclosure provides that valves of the deep veins of the calf (anterior, posterior tibial, and peroneal veins) are recellularized.

The present disclosure provides a method of recellularization of valves in veins by introducing a population of endothelial cells and/or smooth muscle cells, and/or progenitor cells for endothelial and/or smooth muscle cells to the lumen of decellularized veins. The population of cells and/or progenitor cells is cultured with the decellularized veins, thereby recellularizing the valves in the veins. The recellularized valves of the present disclosure restore normal closure time (i.e., competence) and tolerate reflux pressure of similar to normal healthy valve.

In the method of valve recellularization a population of cells, present or derived from peripheral venous blood or whole blood, are cultured with decellularized veins. The present disclosure provides that population of cells is allogeneic. The present disclosure provides that population of cells is autologous. The population of cells is collected from or is present in peripheral venous blood or whole blood. The present disclosure provides that peripheral venous blood or whole blood is from an allogeneic donor. The present disclosure provides that peripheral venous blood or whole blood is autologous. The present disclosure provides that decellularized veins are cultured with allogeneic peripheral venous blood or allogeneic whole blood. The present disclosure provides that decellularized veins are cultured with allogeneic whole blood. The present disclosure provides that decellularized veins are cultured with autologous peripheral venous blood or autologous whole blood.

The present disclosure provides decellularized veins. The veins are decellularized in a method in which the decellularization step is repeated more than once (defined as “cycles”). The present disclosure provides that veins decellularization involves between 2-16 cycles. The present disclosure provides that vein decellularization method involves 14 cycles.

The veins after decellularization in a method involving 2-16 decellularization cycles, are without nuclei (stains negative for nuclei in an immuohistochemical assay), have reduced or no HLA class-I antigen (stains less or no HLA class-I antigen in an immuohistochemical assay), and have reduced or no HLA class-II antigens (stains less or no HLA class-II antigen in an immuohistochemical assay). After 2-16 decellularization cycles, the veins are characterized as having decreased collagen and sulfated glycosaminoglycans (GAG), and increased elastin compared to veins that are not decellularized. The decellularization decreases DNA in decellularized veins compared to veins that are not decellularized.

The present disclosure provides valve recellularization in decellularization veins, in which the recellularized valves have nuclei and are CD31 positive. The veins with recellularized valves have spindle shaped smooth muscle cells in the tunica adventitia of the vein, and are positive for endothelial marker vWF.

The present disclosure provides recellularizing valves in decellularized veins, by a method that achieves functional valves. The functional valves have normal closure time (i.e., competent) and tolerate normal level of reflux pressure.

The present disclosure provides recellularized valves, which restores pressure of 12 to 18 mm Hg at the venous end of the capillary during walking after grafting in a subject in need thereof. In a non-moving subject (e.g., sitting) the recellularized valves of the present disclosure achieves lower extremity venous pressure of about 100 mm Hg (depending on height). The present disclosure provides recellularized valves that restore normal hydrostatic and gravitational pressures after grafting in a subject in need thereof, which are expressed as a constant multiplier (0.77 mm Hg/cm) of the vertical distance in cm below the atrium. Upon grafting, the recellularized valves restore normal highest level of pressure in the upright (sitting or standing) but motionless subject, in need thereof.

The present disclosure provides that the recellularized valves achieve normal level of reflux pressure of about 100 mm Hg, in an in vitro flow circuit.

The present disclosure provides recellularization of valves for achieving normal venous return of blood from the dependent lower extremity to the heart, by ejecting blood by the lower extremity muscle pumps, assisted by the competent recellularized venous valves. The recellularized valves of the present disclosure function to divide the hydrostatic column of blood into segments and prevent retrograde venous flow. The present disclosure provides recellularized valves for use in restoring the greater number of valves in the infrapopliteal segment. The present disclosure provides recellularized valves that for use in ameliorating symptoms resulting from hydrostatic pressure alteration by correcting femoral or popliteal vein incompetence. The present disclosure provides that recellularized perforating vein valves prevent deep to superficial flow, and restores normal pressure/flow relationships of the calf pump. The present disclosure provides that recellularized valves in the perforating veins of the foot restore bidirectional flow of blood through the veins.

The present disclosure further provides a method of treating or ameliorating symptoms of chronic venous insufficiency (CVI) and/or leg ulceration in a subject in need thereof, involving grafting recellularized valve-bearing segments of veins to the subject. The recellularization method includes one or more steps. The method includes one or more steps: harvesting valve-bearing segments of human allogeneic femoral veins; decellularizing the veins in the femoral veins in 2-16 cycles; collecting blood from the subject; perfusing the decellularized veins with an anticoagulant; perfusing the decellularized veins with the collected blood for several hours; draining the blood and rinsing the vein with a buffer; and perfusing the veins first with endothelial cell media for more than one day, and then with smooth muscle media for more than one day; and thereby recellularizing the decellularized valves; wherein the recellularized valve upon grafting to the subject treats or ameliorates CVI and/or leg ulceration.

The valve decellularization steps of the present disclosure comprise treating the valve-bearing veins in a detergent and an organophosphate compound. The valves are decellularized in Triton and Tri-n-butyl phosphate and DNAase.

The decellularized valves are recellularized by perfusing the veins in endothelial cell media for between 2-6 days, followed by perfusing the veins in smooth muscle media for between 2-6 days. In one aspect, the culturing of the decellularized veins is in vitro.

The recellularized valves are CD31 positive. The recellularized valves have mechanical properties of force at first peak above 0.8 N.

The present disclosure provides methods of treating recurrent leg cancer, which is due to deep venous reflux and/or venous hypertension, with recellularized valve-bearing veins. The present disclosure provides use of recellularized valve-bearing veins in treating recurrent leg cancer, which is due to deep venous reflux and/or venous hypertension.

The present disclosure features valves produced by any one of the methods described herein, wherein the recellularized valves are CD31 positive. The recellularized valves can also be smooth muscle actin positive, vWF positive, and/or be characterized by the presence of spindle-shaped smooth muscle cells. The recellularized valves have mechanical properties of force at first peak above 0.8 N.

The present disclosure also features the use of a valves produced by any one of the methods described herein for implantation or grafting, wherein the recellularized valves are CD31 positive. The recellularized valves can also be smooth muscle actin positive, vWF positive, and/or be characterized by the presence of spindle-shaped smooth muscle cells. The recellularized valves have mechanical properties of force at first peak above 0.8 N.

The present disclosure provides recellularization of valves, which upon grafting in a subject in need thereof, treats and/or ameliorates the symptoms of incompetent valves in the thigh. In one aspect, the treatment and/or amelioration of the symptoms is achieved by restoring normal working relationship between muscle pumps and the venous valves. The muscular pumps of the lower limb include those of the foot, calf, and thigh. Among these, the calf pump is the most important as it is most efficient, has the largest capacitance and generates the highest pressures (200 mm of mercury during muscular contraction). The normal limb has a calf volume ranging from 1500 to 3000 cc, a venous volume of 100 to 150 cc, and ejects over 40% to 60% of the venous volume with a single contraction.

During contraction, the gastrocnemius and soleus muscles drive blood into the large capacity popliteal and femoral veins. The recellularized valves of the present disclosure prevent retrograde flow (reflux) during subsequent relaxation, generating negative pressure and drawing blood from the superficial to the deep system through competent perforating veins. The recellularized valves incrementally lower venous pressure until arterial inflow equals venous outflow. The present disclosure provides that when exercise ceases in a subject, the veins with recellularized valves slowly fill the capillary bed, causing a slow return to the resting venous pressure.

Although muscle surrounds the thigh veins, the contribution of thigh muscle contraction to venous return is minimal compared with the calf muscle pump. Pumping action due to compression of the planter venous plexus during ambulation primes the calf pump. Various leg pumps work together with competent valve function to return venous blood from the distal to proximal extremity. The recellularized valves of the present disclosure are for use in restore functional leg pumps to return venous blood from the distal to proximal extremity.

### Method of Decellularization

A total of twelve specimens from cadavers were harvested and tested for function by measuring valve closure time and tolerance to reflux pressure. The median time between death-harvest was 3 days (2-6 days) and death-test 6 days (5-7 days). Prior to transportation of the samples to Sweden, normal closure time (≤0.5 seconds) was registered in 8 of 12 of the cadaver specimens at a pressure of 100 mmHg. Two vein segments did not show normal closure time. Furthermore, two of 10 functional specimens transported, showed mechanical damage in the valves already prior to start of decellularization and remained incompetent after recellularization. Median diameter of the vein specimens was 9.8 mm (7.5-14) and 9.8 mm (8-14.2) after recellularization.

The present disclosure provides for methods and materials to decellularize valves in valve-bearing veins. As used herein, “decellularization” refers to the process of removing cells from valves. Effective decellularization is dictated by factors such as tissue density and organization, geometric and biologic properties desired for the end product, and the targeted clinical application. Decellularization of valves with preservation of the ECM integrity and bioactivity can be optimized by those skilled in the art, for example, by choosing specific agents and techniques during processing.

The most effective agents for decellularization will depend on many factors including cellularity, density, lipid content, and thickness of the vein. It should be understood that while most cell removal agents and methods may alter ECM composition and cause some degree of ultrastructure disruption, minimization of these undesirable effects is preferred. One skilled in the art could readily optimize the decellularization process, as described herein, to minimize the disruption of the ECM scaffold.

One or more cellular disruption solutions can be used to decellularize valve-bearing veins. A cellular disruption solution generally includes at least one detergent, such as SDS, PEG, or Triton X. One of the detergents is Triton X. A cellular disruption solution can include water such that the solution is osmotically incompatible with the cells. Alternatively, a cellular disruption solution can include a buffer (e.g., PBS) for osmotic compatibility with the cells. Cellular disruption solution also can include enzymes such as, without limitation, one or more collagenases, one or more dispases (fibronectin, collagen IV, and collagen I cleaving proteases), one or more DNases, or a protease such as trypsin. In some instances, cellular disruption solution also or alternatively can include inhibitors of one or more enzymes (e.g., protease inhibitors, nuclease inhibitors, and/or collegenase inhibitors).

The present disclosure provides that veins are treated sequentially with two or more different cellular disruption solutions. For example, a first cellular disruption solution contains 1% TRITON X-100® (×100, Sigma, Sweden), a second cellular disruption solution contains 1% tri-n-butyl phosphate (TNBP) 28726.1, VWR, Sweden), and a third cellular disruption solution contains 0.004 mg/ml deoxyribonuclease I (DNase I) (D7291, Sigma, Sweden). Sequential treatment may include repeating treatment with at least one of the cellular disruption solutions in the treatment sequence. In some aspects, the vein may be treated by decellularization cycles comprising the sequential treatment of one or more cellular disruption solutions in the same order until the desired level of decellularization is achieved. The present disclosure provides that the number of decellularization cycles is at least 2, at least 3, at least 4, at least 5, at least 6, at least 7, at least 8, at least 9, at least 10, at least 11, at least 12, at least 13, at least 14, at least 15, at least 16, at least 17, at least 19, or at least 20 cycles. The number of cycles needed for desired decellularization is determined through monitoring for presence of nuclei, HLA class I, or class II antigens, and other indications of presence of cells in the veins. Lack of nuclei present on decellularized veins is indicative of the level of decellularization of the veins.

The present disclosure provides that each cellular disruption solution may further comprise additional components, such as antibiotics (i.e., penicillin, streptomycin, and amphotericin), ethylenediaminetetraaceticacid (EDTA) disodium salt dehydrate (EDTA), and/or phenyl methyl sulfonyl fluoride (PMSF). For example, a cellular disruption solution that comprises DNase I may also include calcium chloride and magnesium chloride (A12858, Life Technologies) to activate the enzyme.

Perfusion methods may be used to treat the valve-bearing veins with cellular disruption solutions for decellularization of the vein. Alternating the direction of perfusion (e.g., antegrade and retrograde) can help to effectively decellularize the valve-bearing veins. Decellularization as described herein essentially removes the cells lining the valve-bearing veins from the inside out, resulting in very little damage to the ECM. Depending upon the size and weight of the tissue and the particular detergent(s) and concentration of detergent(s) in the cellular disruption solution, a valve-bearing vein generally is perfused from about 2 to about 12 hours per gram of tissue with cellular disruption medium. Including washes, an organ may be perfused for up to about 12 to about 72 hours per gram of tissue. Perfusion generally is adjusted to physiologic conditions including pulsatile flow, rate and pressure. Perfusion decellularization as described herein can be compared to immersion decellularization as described, for example, in U.S. Pat. Nos. 6,753,181 and 6,376,244.

The present disclosure provides that valve-bearing veins are filled with cellular disruption solutions and simultaneously agitated for decellularization of the veins. Different cellular disruptions solutions are added in a sequential order and the order repeated multiple times until the desired level of decellularization is achieved. For example, one end of the vein is kept open while the rest of the openings (i.e., abrasions and branches) were sutured to prevent leakage. The vein is first rinsed in PBS containing antibiotics (0.5% penicillin, 0.5% streptomycin and 0.5% amphotericin B). Then the vein is rinsed in distilled water for 72 hours. Each decellularization cycle preferably consists of incubation with 1% Triton X for 3 hours, followed by 1% TnBP for 3 hours, and 0.004 mg/ml DNase I for three hours. Lastly, the vein is washed with distilled water overnight to remove cell debris. In each incubation period, the vein is filled with the cellular disruption solution and is clamped closed. Then the vein is placed on an agitator at 37° C. for the incubation time (3 hours or overnight) with gentle shaking. At the end of each incubation period, the contents of the valve-bearing veins are removed and the vein is rinsed with PBS. After 7-9 cycles (of TRITON X®, TnBP, DNaseI and water wash) plus agitation, the vein is washed continuously for 48 hours with PBS, where the PBS was replaced every 6 hours. Varying concentrations of detergent (TRITON X® or TnBP) can be utilized, as needed or to the discretion of one ordinarily skilled in the art. Varying concentrations of enzymes, such as DNase, can be utilized, as needed or to the discretion of one ordinarily skilled in the art.

The present disclosure provides sterilizing decellularized valve-bearing veins prior to recellularization steps. For example, the sterilization process involves incubating the decellularized veins in 0.1% peracetic acid in sterile PBS for 1 hour, followed by washing with sterile water and PBS for 4 hours with each solution.

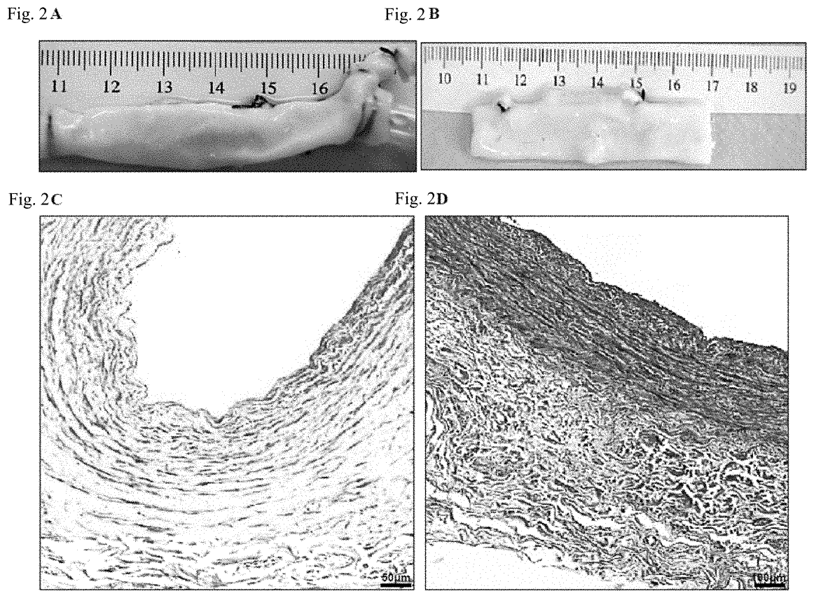

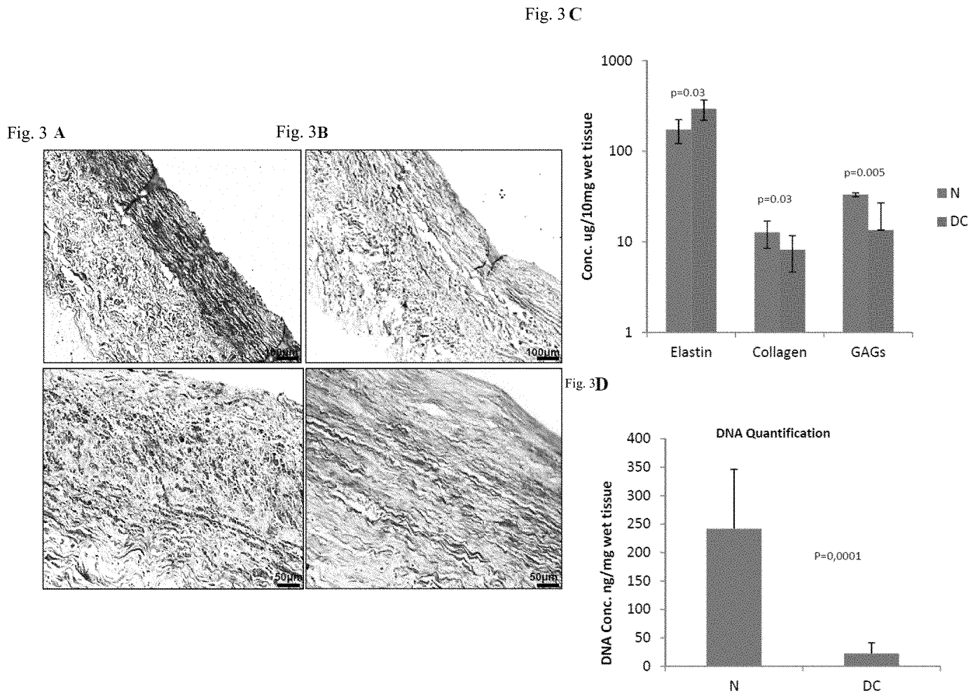

The present disclosure provides treating valve-bearing veins with a suitable detergent, e.g., TRITON®, and a solvent/detergent (e.g., 2-percent tri(n-butyl)phosphate (TNBP)) and optionally treating with DNAase to decellularize the veins. Valve-bearing veins are recellularized by treating with detergent, e.g., TRITON®, and a solvent/detergent (e.g., 2-percent tri(n-butyl)phosphate (TNBP)) and optionally treating with DNAase, in 2-16 cycles. The present disclosure provides that valve-bearing veins are treated in detergent and a solvent/detergent in 14 cycles. The method of decellularization of valve-bearing veins does not result in leakage or changes in size of the veins after decellularization. The decellularization of valve-bearing veins results in the loss of nuclei in the veins (see FIG. 2C) after 14 cycles. At initial cycles of decellularization, veins have abundant amount of collagen but no nuclei (see FIGS. 3A&B).

The present disclosure provides a method of decellularization of valve-bearing veins, by which the decellularized veins are characterized as negative for HLA class-I or -II antigens. The present disclosure provides a decellularization method which results in significant loss of collagen and GAGs of the extracellular matrix (ECM), while the level of elastin in the ECM is increased. The decellularized valve veins of the present disclosure results in significant loss of collagen and GAGs, and significant increase in elastin as compared to normal (i.e., non-decellularized) veins. The decellularization protocol of the current disclosure leads to significant decrease in DNA amount in decellularized veins (e.g., from 241.95±39.44 ng/mg of tissue in normal veins to 22.44±6.29 ng/mg in decellularized veins).

To effectively recellularize and generate an allogeneic valves, it may be important that the morphology and the architecture of the ECM be maintained (i.e., remain substantially intact) during and following the process of decellularization. “Morphology” as used herein refers to the overall shape of the organ or tissue or of the ECM, while “architecture” as used herein refers to the exterior surface, the interior surface, and the ECM there between. The morphology and architecture of the ECM can be examined visually and/or histologically to verify that the decellularization process has not compromised the three-dimensional structure and bioactivity of the ECM scaffold. Histological analysis by staining (i.e., H&E, MT or VVG) is useful to visualize decellularized vein architecture and preservation of ECM components, such as collagen I, collagen IV, laminin and fibronectin. Other methods and assays known in the art may be useful for determining the preservation of ECM components, such as glycosaminoglycans and collagen. Importantly, residual angiogenic or growth factors remain associated with the ECM scaffold after decellularization. Examples of such angiogenic or growth factors include, but are not limited to VEGF-A, FRF-2, PLGF, G-CSF, FGF-1, Follistatin, HGF, Angiopoietin-2, Endoglin, BMP-9, HB-EGF, EGF, VEGF-C, VEGF-D, Endothelin-1, Leptin, and other angiogenic or growth factors known in the art.

### Method of Recellularization

The present disclosure provides for materials and methods for generating regenerated valves in veins. A regenerated valve can be produced by contacting a decellularized vein from a donor as described herein with a population of cells in the lumen of the vein and culturing said population of cells in the lumen of the decellularized vein. The present disclosure provides culturing a decellularized vein, where valves in the vein are also decellularized, with whole blood or peripheral blood. The present disclosure provides that decellularized veins are perfused with whole blood or peripheral blood. The whole blood or the peripheral blood is from the subject in need for treating or ameliorating a disease and/or disorder due to an impaired or dysfunctional valve.

As used herein, “recellularization” refers to the process of introducing or delivering cells to a decellularized vein and culturing the cells such that the cells proliferate and/or differentiate eventually to regenerate a valve with architecture, cell organization, and bioactivity similar to that of normal valve-bearing vein.

The present disclosure includes a method of recellularization of a valve in a vein with a small amount (e.g., 10 mL-30 mL) of whole blood or peripheral venous blood. In embodiments, the valve is recellularized without in vitro culture, expansion, and differentiation of endothelial cells progenitor cells and smooth muscle progenitor cells in culture plate. In embodiments, the decellularized valves may be recellularized, after perfusing with blood, by perfusing the veins in endothelial cell media for between 2-6 days, followed by perfusing the veins in smooth muscle media for several days, e.g., 2-6 days. In embodiments, the culturing of the decellularized veins may be in vitro or ex vivo. In embodiments, the culturing of the decellularized veins may be in a bioreactor.

The present disclosure provides a recellularization method in which peripheral venous blood is collected and placed in a container coated with an anticoagulant (i.e., heparin coated vacutainer tubes) and transported to the laboratory as soon as possible (e.g., within about 2 hours). The volume of blood required can vary depending on the length of the vein and of the pipes used in the bioreactor in which the recellularization is performed.

The recellularization process is performed with perfusions carried out in an incubator at about 37° C. supplied with 5% CO₂. Before recellularization, the veins are perfused with an anticoagulant (e.g., heparin). After washing off the anticoagulant, the vein is perfused for several hours at a desired speed (e.g., for about 48 hours at about 2 ml/min). The perfusion is continued for about 2 or more days (e.g., for about 4 days) with endothelial cell media and then another about 2 or more days (e.g., for 4 days) with smooth muscle cell media. The number of days perfusion is carried out is an optional feature, which can be adjusted depending on the cells or whole blood and the cellularization efficacy.

The complete endothelial medium may be prepared using MCDB131 (Life technologies, Sweden) basal medium supplemented with about 10% heat inactivated human AB serum (Life technologies, Sweden), about 1% glutamine (Lonza, Denmark), about 1% penicillin-streptomycin-amphotericin, and EGM2 single quote kit (Lonza, Denmark) that contained ascorbic acid, hydrocortisone, transferrin, insulin, recombinant human VEGF, human fibroblast growth factor, human epithelial growth factor, heparin and gentamycin sulfate. The complete smooth muscle medium may be prepared using about 500 ml Medium 231 (Life technologies, Sweden) supplied with about 10% heat inactivated human AB serum, about 1% penicillin-streptomycin amphotericin and about 20 ml smooth muscle growth supplement (SMGS) (Life Technologies, Sweden). Other media for growing endothelial cells and/or smooth muscle cells that are well known in the art may also be used. Vein scaffolds were recellularized for a total of about ten days.

The present disclosure provides that population of cells utilized for recellularization are derived from stem or progenitor cells, for example, bone-marrow-derived stem or progenitor cells, or cells expressing CD133 (CD133+ cells). Stem or progenitor cells can be expanded and differentiated in vitro into endothelial cells and/or smooth muscle cells by methods known in the art. For example, stem or progenitor cells can be cultured in the presence of certain growth factors and supplements that initiate differentiation into endothelial cells and/or smooth muscle cells. In some aspects, the differentiated cells may not be terminally differentiated, but express at least one endothelial cell marker (i.e., CD31 or vWF) or at least one smooth muscle cell marker (i.e., smooth muscle actin) prior to introduction to the decellularized vein. The endothelial cells and smooth muscle cells derived from the stem or progenitor cell as described herein are introduced to the decellularized vein, for example, by perfusion. Culturing of the endothelial cells and smooth muscle cells comprise incubating the cells and vein with endothelial cell medium or smooth muscle cell medium in alternating cycles until the desired recellularization is achieved.

Post natal vasculogenesis is the formation of new veins in adults by circulating endothelial progenitor cells (EPCs); and angiogenesis is formation of new veins from pre-existing endothelial cells (Ribatti D et al., 2001). These two processes contribute in formation of vein branches and in pathogenic states like wound healing, ischaemia, fracture healing, tumor growth etc., (Laschke et al., 2011). There are endothelial cells and endothelial progenitor cells co-existing in circulation in whole blood, and the endothelial progenitor cells contribute to vascularization (Asahara T et al., 1997). Furthermore, progenitor cells for smooth muscle cells are also present in circulating whole blood (Simper D et al., 2002).

The present disclosure provides that population of cells utilized for recellularization is from whole blood. Use of whole blood for regeneration of a decellularized vein, would result in efficient recellularization of veins without the need to isolate and expand subpopulations of angiogenic progenitor cells from bone-marrow or whole blood. Whole blood is introduced to the decellularized vein, for example, by perfusion.

There are many advantages of the present disclosure over the options for vascular grafts currently available. The present disclosure provides an autologous engineered vein with the following advantages: 1) is non-immunogenic and therefore having minimal risk of graft rejection or adverse immune response; 2) obviates the need for immunosuppression, and therefore less risk to the patient after surgery and for their lifetime; 3) has no length restriction; 4) is more readily available, as compared to matched donor veins or autologous veins; 5) is composed of natural components (i.e., ECM, endothelial cells and smooth muscle cells), and therefore has superior qualities to mostly synthetic and artificial veins, including preserving residual angiogenic growth factors and biomechanical integrity; 6) production of vein is minorly invasive in comparison to harvesting autologous vein for transplant; 7) use of whole blood cells allows rapid and minimally invasive procedure to subject.

The population of cells as used herein may be any cells used to recellularize a decellularized valve-bearing vein. These cells can be totipotent cells, pluripotent cells, or multipotent cells, and can be uncommitted or committed. In addition, cells useful in the present disclosure can be undifferentiated cells, partially differentiated cells, or fully differentiated cells. Cells useful in the present disclosure also include progenitor cells, precursor cells, and “adult”-derived stem cells. Examples of cells that can be used to recellularize a vein include, but are not limited to, bone marrow-derived stem or progenitor cells, bone marrow mononuclear cells, mesenchymal stem cells (MSC), mutltipotent adult progenitor cells, whole-blood derived stem or progenitor cells such as endothelial stem cells, endothelial progenitor cells, smooth muscle progenitor cells, whole blood, peripheral blood, and any cell populations that can be isolated from whole blood. The progenitor cells are defined as cells that are committed to differentiate into one type of cells. For example, endothelial progenitor cells means cells that are programmed to differentiate into endothelial cells; smooth muscle progenitor cells means cells that are programmed to differentiate into smooth muscle cells. The present disclosure provides progenitor cells in whole blood or peripheral blood including population of uncommitted and/or committed cells, such as pluripotent cells or totipotent cells, for use in cellularizing decellularized valve-bearing veins. A feature of the present disclosure is cellularizing decellularized valve-bearing veins with whole blood.

The present disclosure provides that population of cells used to recellularize the vein is allogeneic. “Allogeneic” as used herein refers to cells obtained from the same species as that from which the organ or tissue originated (i.e., self or related or unrelated individuals.). The present disclosure provides that cells or whole blood are from the recipient or the subject in need of treatment (i.e., “autologous”).

The population of cells may be a heterogeneous population of cells. For example, the cells may be whole blood cells, or from whole blood. These cells include red blood cells, white blood cells, thrombocytes, endothelial cells, endothelial progenitor cells, and smooth muscle progenitor cells. It is known in the art that circulating endothelial cells, endothelial progenitor cells, and progenitor cells for smooth muscle cells can contribute to vasculogenesis and angiogenesis. Thus, application of whole blood cells can readily supply a decellularized vein with cells capable of expanding and differentiating into endothelial and smooth muscle cells for the regeneration of the vein.

The population of cells utilized for recellularization may be isolated from a heterogeneous population of cells. The present disclosure provides that population of cells may be stem or progenitor cells isolated from bone marrow. The present disclosure provides that population of cells may be endothelial cells or endothelial progenitor cells (i.e., committed cells) isolated from whole blood. Methods for isolating particular populations of cells from a heterogeneous population are known in the art. Such methods include lymphotrap, density gradients, differential centrifugation, affinity chromatography, and FACS flow cytometry. Markers known in the art that identify particular populations of cells of interest may be used to isolate the cells from the heterogeneous population. For example, CD133 is known to be expressed on the surface of stem cells or stem-like cells derived from the bone marrow. Selection for CD133+ cells can be achieved by utilization of MACs beads and specific antidbodies that recognize CD133. Markers specific for endothelial progenitor or smooth muscle cell progenitor cells can also be utilized to purify the population of cells of interest.

In some aspects, the population of cells may be cultured in vitro prior to introduction to the decellularized vein. The purpose of culturing in vitro includes expanding cell numbers and differentiating cells to specific cell lineages of interest. The present disclosure provides that population of cells may be first isolated from a heterogeneous population prior to culturing in vitro. The present disclosure provides that population of cells may be bone marrow-derived stem or progenitor cells (i.e CD133+ cells) and may be differentiated in vitro prior to introduction to the decellularized vein. Various differentiation protocols are known in the art and include, for example, growing cells in growth media supplemented with factors, agent, molecules or compounds that induce differentiation into endothelial cells or smooth muscle cells.

The number of cells that is introduced to a decellularized vein in order to generate a vein may be dependent on the size (i.e., length, diameter, or thickness) of the vein and the types of cells used for recellularization (i.e., stem cells vs. more differentiated cells, such as whole blood). Different types of cells may have different tendencies as to the population density those cells will reach. By way of example, a decellularized organ or tissue can be “seeded” with at least about 1,000 (e.g., at least 10,000, 100,000, 1,000,000, 10,000,000, or 100,000,000) cells; or can have from about 1,000 cells/mg tissue (wet weight, i.e., prior to decellularization) to about 10,000,000 cells/mg tissue (wet weight) attached thereto.

The population of cells can be introduced (“seeded”) into a decellularized vein by injection into one or more locations. In addition, more than one type of cell (i.e., endothelial cells or smooth muscle cells) can be introduced into a decellularized vein. For example, The present disclosure provides that both endothelial cells and the smooth muscle cells are introduced to the lumen of the decellularized vein. Alternatively, or in addition to injection, the population of cells can be introduced by perfusion into a cannulated decellularized vein. For example, the population of cells can be introduced to a decellularized vein by perfusion. After perfusion of the cells, expansion and/or differentiation media may be perfused through the vein to induce growth and/or differentiation of the seeded cells. The present disclosure provides that anti-coagulant agents, such as heparin, may be administered prior to and/or simultaneously to the introduction the population of cells.

Expansion and differentiation media, as used in the present disclosure, includes cell growth medium containing supplements and factors required for proliferation of endothelial cell or smooth muscle cell, and differentiation to endothelial cell or smooth muscle cell. The present disclosure provides that differentiation medium for endothelial cells may be the same as the growth/proliferation medium for endothelial cells. For example, additional factors or supplements present in endothelial growth or differentiation media may include, but are not limited to: ascorbic acid, hydrocortisone, transferrin, insulin, recombinant human VEGF, human firbroblast growth factor, human epithelial growth factor, heparin and gentamycin sulfate. The present disclosure provides that differentiation medium for smooth muscle cells may be the same as the growth/proliferation medium for smooth muscle cells. For example, additional factors or supplements present in endothelial growth or differentiation media may include, but are not limited to: smooth muscle growth supplement, smooth muscle differentiation supplement, MesenPro, and transforming growth factor β1. At minimum, growth and differentiation media comprise a base media (i.e., MCDB131, M231, or DMEM) heat inactivated serum (for example, at 10%), glutamine and antibiotics (i.e., penicillin, streptomycin, amphotericin).

The present disclosure provides incubating or perfusing seeded vein with endothelial cell media and smooth muscle cell media in alternation until achieving the desired recellularization. The present disclosure provides repeating perfusion of endothelial cell media and smooth muscle cell media multiple times and in alternation, for example, at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times or at least 15 times. The present disclosure provides that duration of perfusion of endothelial cell media may be the same as the duration of perfusion of smooth muscle cell media. Alternatively, the duration of perfusion of endothelial cell media may be different from the duration of perfusion of smooth muscle cell media. Duration of perfusion of either differentiation or growth media may be dependent on the characteristics of the population of cells seeded on the decellularized vein. Duration of perfusion of the differentiation and growth media may be determined by one skilled in the art.

During recellularization, the decellularized vein may be maintained under conditions in which at least some of the seeded cells can multiply and/or differentiate within and on the decellularized vein. Those conditions include, without limitation, the appropriate temperature and/or pressure, electrical and/or mechanical activity, force, the appropriate amounts of O₂ and/or CO₂, an appropriate amount of humidity, and sterile or near-sterile conditions. During recellularization, the decellularized vein and the cells attached thereto are maintained in a suitable environment. For example, the cells may require a nutritional supplement (e.g., nutrients and/or a carbon source such as glucose), exogenous hormones or growth factors, and/or a particular pH.

The present disclosure also provides for a bioreactor for recellularizing a vein under the appropriate conditions, as described herein. Specifically, the bioreactor comprises a completely closed chamber that is large enough to fit the vein to be recellularized and can be sterilized, a tube for supplying cells and/or media connected to a pumping mechanism (i.e., a peristaltic pump), a structure to which one end of the vein is connected to, and 2 inlets and 2 outlets. The set-up of the tubes in relation to the vein and pump allows the cells or media to flow through the lumen of the vein, and flow around, or immerse, the exterior of the vein.

In some instances, a vein generated by the methods described herein is to be transplanted into a patient. In those cases, the cells used to recellularize a decellularized vein can be obtained from the patient such that the regenerative cells are “autologous” to the patient. Cells from a patient can be obtained from, for example, blood, bone marrow, tissues, or organs at different stages of life (e.g., prenatally, neonatally or perinatally, during adolescence, or as an adult) using methods known in the art. Alternatively, cells used to recellularize a decellularized organ or tissue can be syngeneic (i.e., from an identical twin) to the patient, the cells can be human lymphocyte antigen (HLA)-matched cells from, for example, a relative of the patient or an HLA-matched individual unrelated to the patient, or cells can be allogeneic to the patient from, for example, a non-HLA-matched donor.

The progress of the seeded cells can be monitored during recellularization. For example, the number of cells on or in the decellularized vein or tissue can be evaluated by taking a biopsy at one or more time points during recellularization. In addition, the amount of differentiation that the cells have undergone can be monitored by determining whether or not various markers are present in a cell or a population of cells. Markers associated with different cells types and different stages of differentiation for those cell types are known in the art, and can be readily detected using antibodies and standard immunoassays, immunofluorescence, immunohistochemistry or histology techniques. For example, to confirm the presence of endothelial cells, or cells that have differentiated in the endothetlial lineage, any endothelial markers known in the art can be assayed. The endothelial markers include, but are not limited to CD31, vWF, VE-cadherin and AcLDL. For example, to confirm the presence of smooth muscle cells, or cells that have differentiated in the smooth muscle cell lineage, any smooth muscle cell markers known in the art can be assayed. The smooth muscle cell markers include, but are not limited to smooth muscle actin and vimentin.

The present disclosure provides that tensile strength of the engineered vein may be tested. Tensile strength tests are known in the art. For example, an engineered vein may be cut laterally into ring segments and tested by radial deformation. Total force used to break the samples completely and elongation at 50% total force can be calculated to determine tensile strength. The present disclosure provides recellularized veins, which demonstrate increased tensile strengths when compared to decellularized veins. For example, engineered veins of the present disclosure may demonstrate the ability to withstand 10%, 20%, 30%, 40%, 50%, 60%, 70%, 80%, 90%, 100% or more total force in comparison to decellularized veins. In a feature of the present disclosure, the recellularized veins demonstrate similar, or about the same tensile strength as normal veins.

The present disclosure provides valve-bearing segments of human allogeneic femoral veins that harvested from cadavers. The segments are tissue-engineered using the technology of decellularization and recellularization. The de- and recellularized vein (DV/RV) segments are characterized biochemically, immuno-histochemically and biomechanically. Valve closure time (at 100 mmHg) and resistance to reflux pressure is measured in an in vitro model. DNA quantification of DV verified that cellular components are satisfactorily removed. The resultant DV retained some of the extracellular matrix proteins and mechanical integrity. Valve mechanical parameters are similar to the native tissue even after de- and recellularization. The lumen of the scaffold including the valves is successfully re-seeded with endothelial and smooth muscle cells in a bioreactor.

### Characterization of Recellularized Valves

The decellularization protocol of the current disclosure successfully preserves the functional properties of the valves. Tissue-engineered veins with valves provide a valid template for future preclinical studies and eventual clinical applications. The present disclosure provides recellularized functional valves after that upon grafting the recellularized valve in a subject can function as a normal or near normal valve. The methods disclosed herein may enable replacement of diseased incompetent deep veins and causal treatment of venous reflux. The de- and recellularized valve-bearing vein (DV/RV) segments are characterized biochemically, immunohistochemically and biomechanically as described herein to confirm the functional capability of the recellularized valve-bearing vein for successful clinical application.

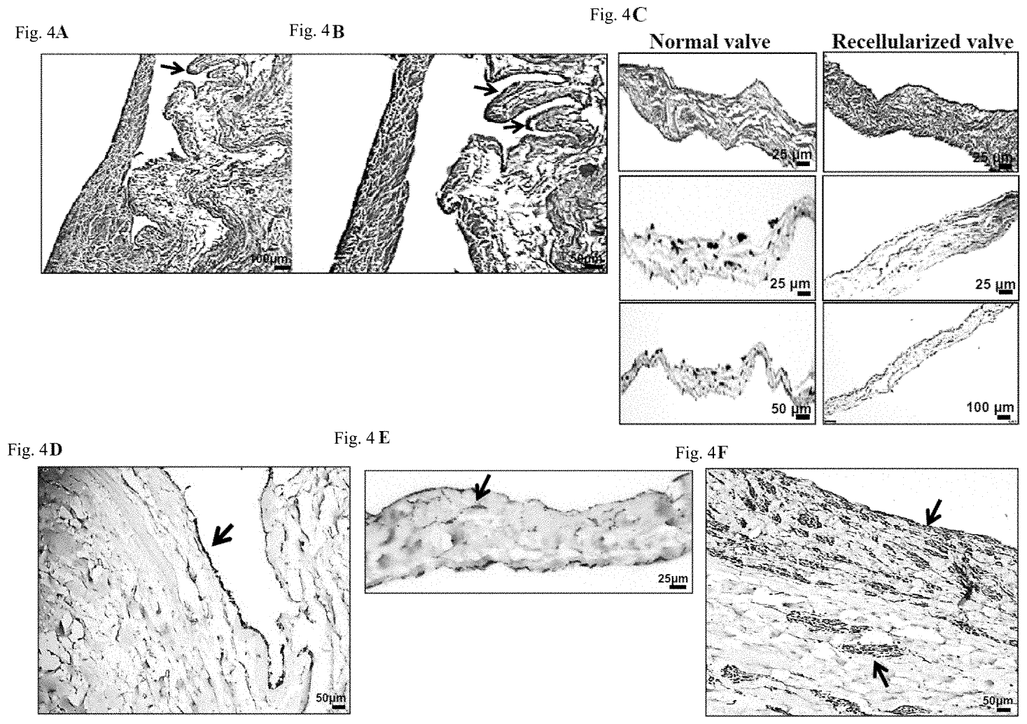

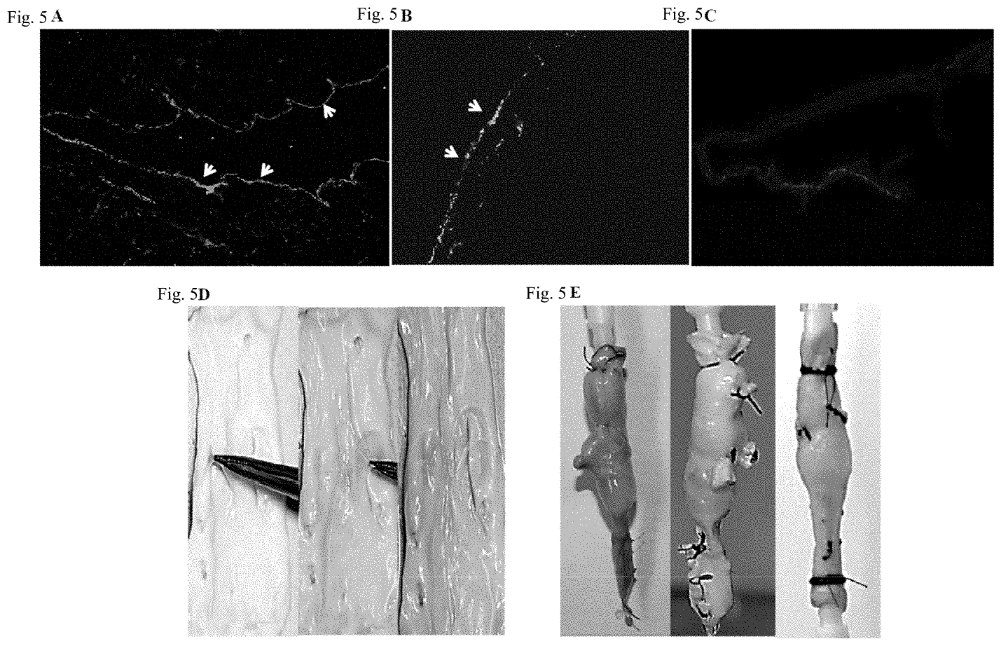

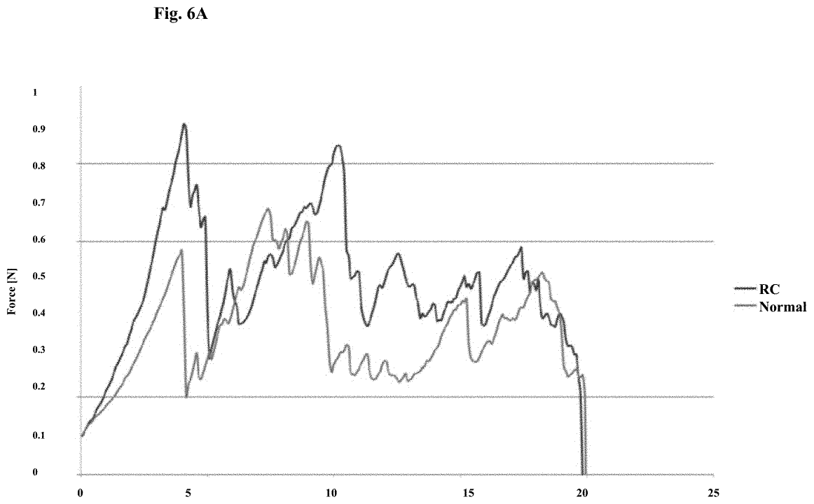

The recellularized valves are characterized for presence of endothelial and smooth muscle cells. Immunohistochemistry and immunofluorescence techniques well known to the ordinarily skilled artisan are utilized to detect the presence or absence of endothelial and smooth muscle cells. To visualize the presence of endothelial cells, antibodies to CD31 (1:200) (Abcam, Germany) and vWF (1:100) (Santa Cruz, Germany) are selected and are used for staining of the recellularized valves. Presence of CD31 and/or vWF positive cells in the recellularized veins are detected by immunohistochemistry and immunofluorescence (see FIGS. 4D, 4E, 5A and 5B). To visualize smooth muscle cells, antibody against smooth muscle actin (1:50) (Abeam, Germany) is used to stain the recellularized valves. Presence of smooth muscle actin positive cells is determined by immunohistochemical analysis (see FIG. 4F). Smooth muscle cells can also be identified by immunohistochemical and immunofluorescence analysis of the morphology of the cells lining the recellularized valve-bearing veins for presence of spindle-shaped muscle cells.

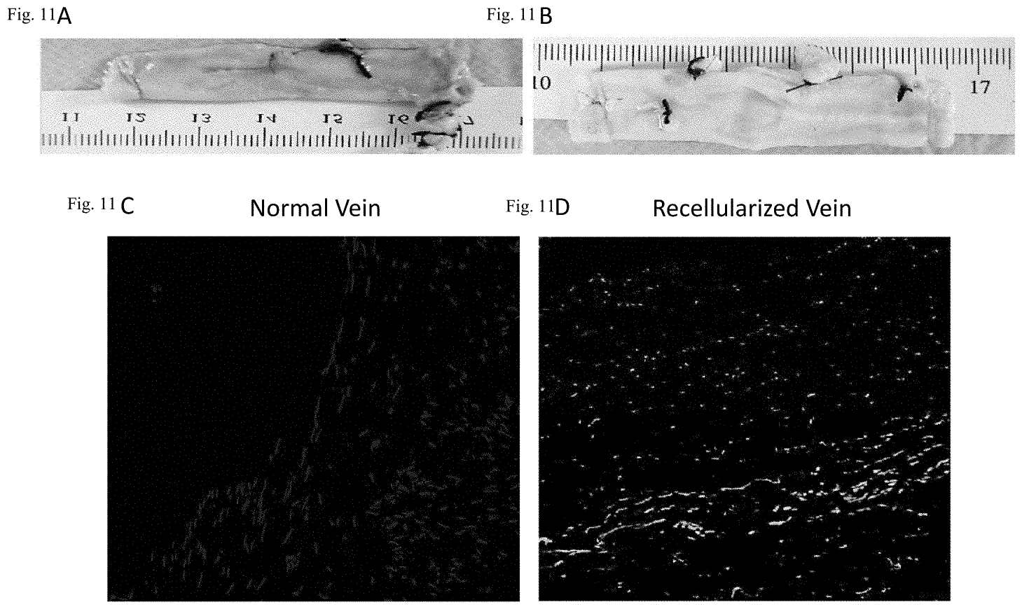

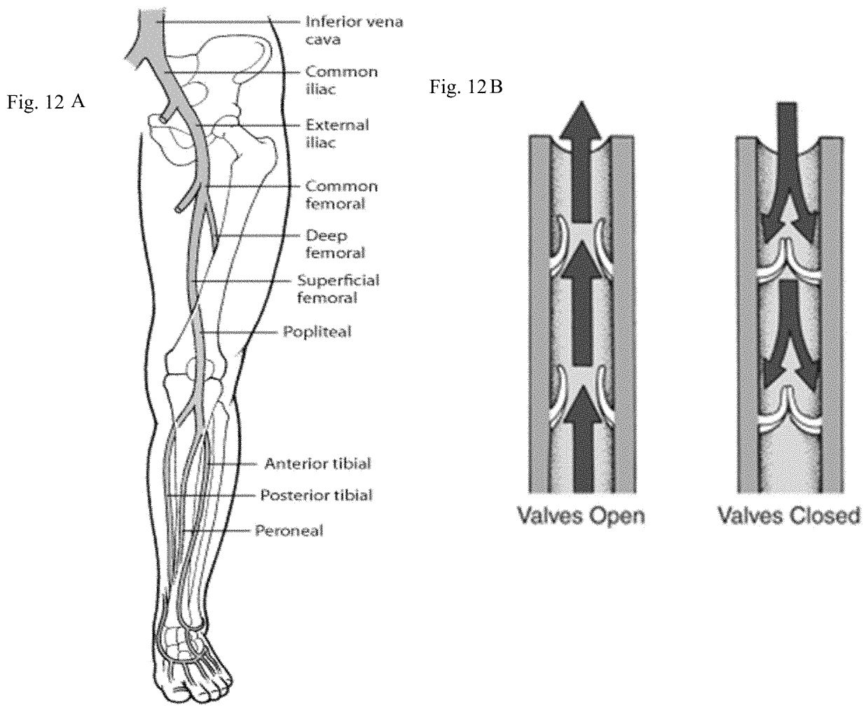

Recellularized valves and veins are also characterized by the presence of nuclei. The recellularized valve-bearing veins are stained by DAPI and staining is visualized by immunofluorescence to detect the presence of nuclei (see FIG. 11D). Immunohistochemical staining, such as Hematoxylin and eosin (HE) staining or Masson's trichrom (MT) staining of the recellularized valves and veins is also suitable for the detection of nuclei (see FIGS. 4A, 4B, and 4C).

Various methods are known in the art for testing valve function. Leakage of the recellularized valve-bearing vein and the recellularized vein is tested by flushing the veins with a solution (i.e., a physiologically buffered solution such as phosphate buffered saline (PBS)). For example, a syringe filled with a solution is inserted into one end of a recellularized vein or valve. The solution is flushed into the valve or vein, preferably at a steady rate, and the surface of the valve or vein is observed for any accumulation of solution resulting from a leak or hole in the recellularized valve or vein. In a preferred feature of the present disclosure, the recellularized valve-bearing vein demonstrates no leakages when tested.

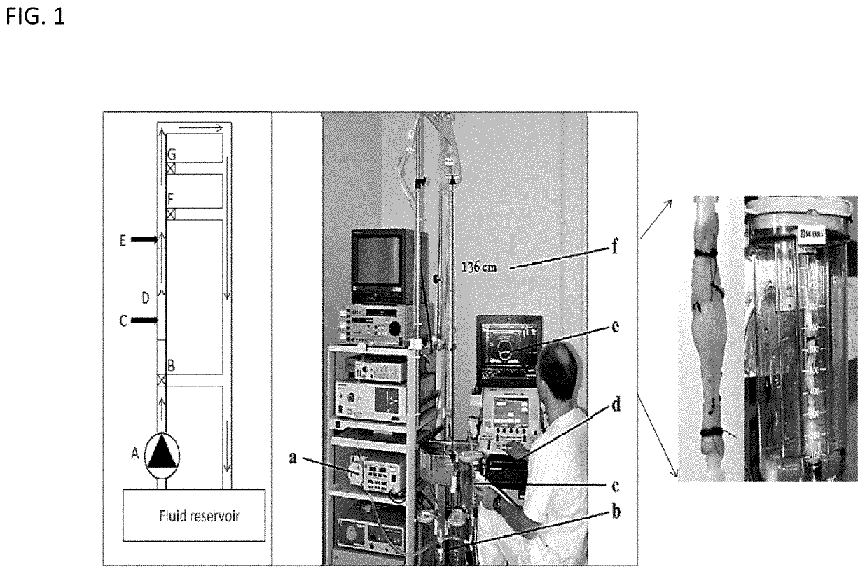

The present disclosure further provides that the function of the recellularized valve-bearing vein can be evaluated by in vitro testing, i.e., by a hydraulic flow system (bioreactor) that mimics physiological flow and pump action. The in vitro test setup used in the present study for evaluating the functionality of the recellularized valve (i.e., competency and reflux pressure tolerance) is a modification to the one used by Geselschap J H, van Zuiden J M, Toonder I M, and Wittens, C H A. In vitro evaluation of a new autologous valve-stent for deep venous incompetence, JOURNAL OF ENDOVASCULAR THERAPY: AN OFFICIAL JOURNAL OF THE INTERNATIONAL SOCIETY OF ENDOVASCULAR SPECIALISTS. (2006), 13:762-769. This model allows the assessment of venous valve function at a determined pressure. To mimic the flow through a vein in the deep venous system under stress, a commercial peristaltic pump was utilized to maintain a constant ante-grade flow, or to delivery intermittent flow to the circuit at the desired pressure levels. The addition of ultrasound contrast Sonovue™ allows the duplex imaging of flow, valve leaflets and valve closure, for the determination of competency (i.e. normal valve closure time). An exemplary bioreactor set-up is illustrated in FIG. 1.

The valve-bearing vein is tested before or after decellularization and/or recellularization. The vein is mounted vertically in the flow circuit and perfused with room-temperature saline. The vein is connected to the fluid circuit by conic connects, secured with sutures at both ends. A mechanical valve is used to regulate flow direction through the circuit during outflow from the peristaltic pump. Ultrasound Doppler technique is used to detect potential reflux at the site of the venous valve.

The competence of a valve, as used herein, refers to valve closure time, or the time from flow reversal until cessation of flow. The valve closure time of a valve with normal function is equal to or less than 0.5 seconds. Thus, in a preferred feature of the present disclosure, the recellularized valves of the present disclosure have a valve closure time (or competence) of equal to or less than 0.5 seconds.

Venous reflux, as used herein, refers to the backward flow of blood against the direction of blood flow toward the heart. A valve with normal function is able to withstand, or tolerate, reflux pressure of about 100 mm Hg and reduced to a mean of about 18 mm Hg to about 25 mm Hg during walking 7 to 12 steps. In other words, a valve with normal function remains closed, thereby preventing reflux, against pressure of up to 100 mm Hg and reduced to a mean of about 18 mm Hg to about 25 mm Hg during walking 7 to 12 steps. The recellularized valve demonstrates the ability to withstand 10%, 20%, 30%, 40%, 50%, 60%, 70%, 80%, 90%, 100% of the reflux pressure tolerated by normal valves, i.e., 100 mm Hg.

The capability of the recellularized valves to function as normal valves can be further tested using various assays to test biomechanical properties of the valve. An instron machine, or other machine for testing tensile strength or shear stress is suitable for measuring the mechanical properties of the recellularized valve.

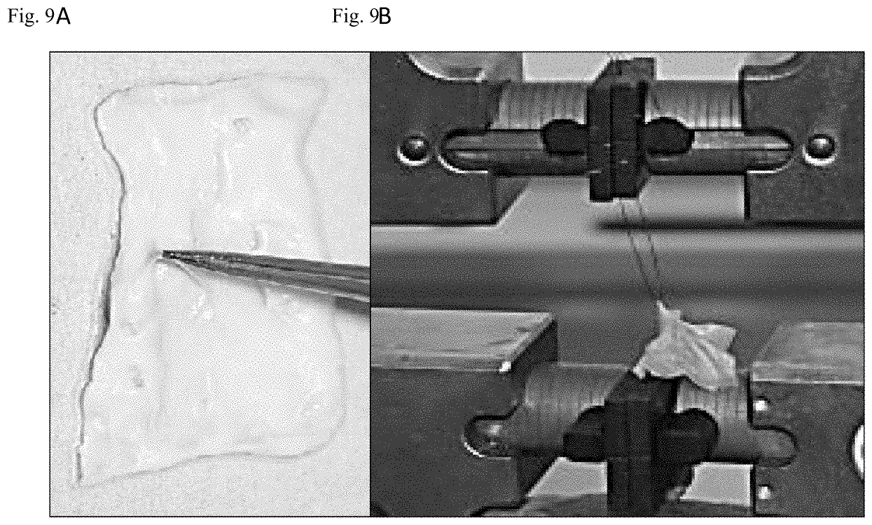

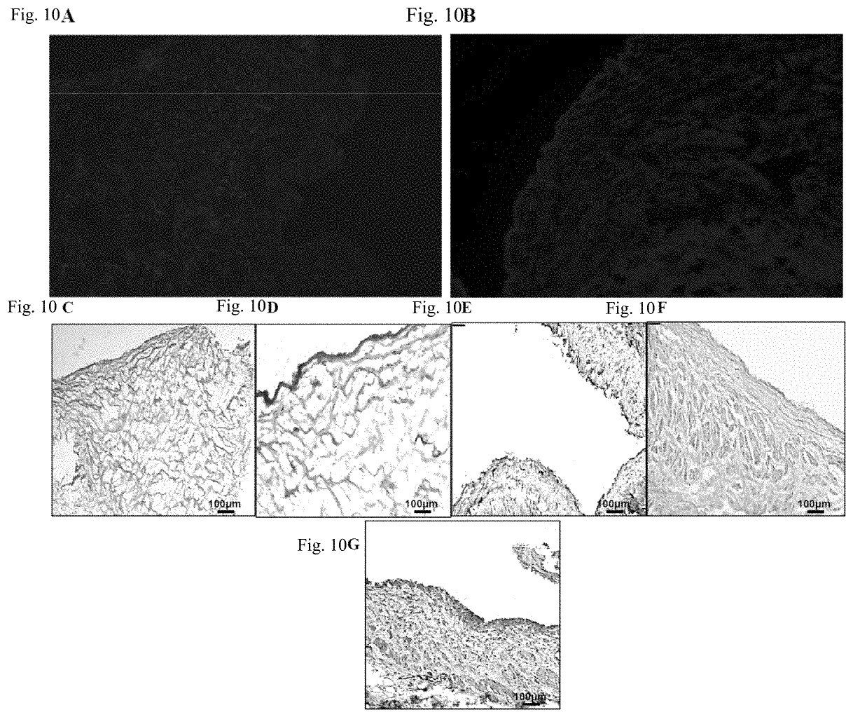

The present disclosure provides evaluation of the mechanical properties of the venous valves by tearing the valve in the horizontal direction. Optionally, sutures can be used to help facilitate the assessment by the machine. Specifically, 4-0 non-absorbable monofilament suture made of polypropylene is attached to the test valve to facilitate loading onto the instron machine (see FIGS. 9A-9B). A predetermined pre-load is used as a starting point for testing, such as 0.1N. A test speed of 20 mm/minute is used to fracture the test valve horizontally. The test valve is extended (mechanically) over time, and the force is measured as a function of the extension distance (i.e., mm). The force withstood by the test vein until the first tear is designated as the first peak. Normal veins with normal function can withstand about 0.8N force at the first peak.

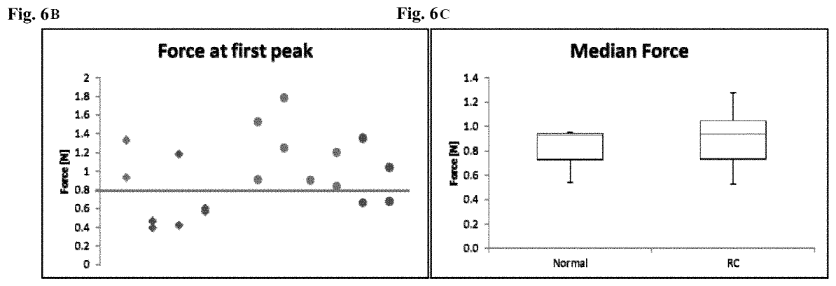

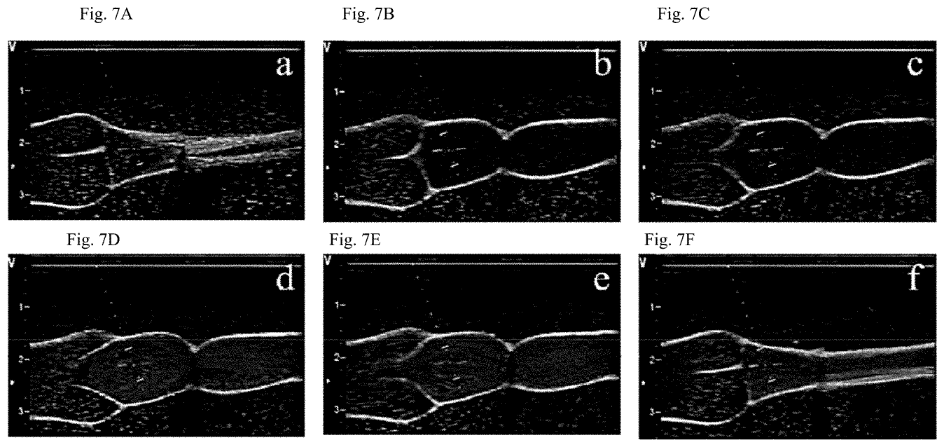

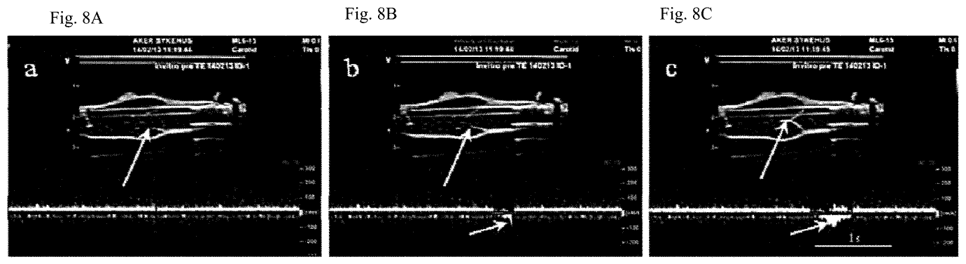

Valve-containing veins were decellularized and recellularized using the methods described herein and tested for its biomechanical properties in comparison to normal veins. Mechanical analysis of the valves showed that 4/6 recellularized valve-bearing veins and 1/4 of the tested normal valve-bearing veins were functional (see FIGS. 6A-6C). In each vein a pair of valves was present and the force at first peak for each pair of valves was measured. Force at first peak was analyzed to find differences between veins that functioned and those that did not. Based on the results obtained, failure of even one of the valves in the pair is sufficient to affect the functionality of the vein. Preferably, the recellularized valves obtained by the methods disclosed herein can withstand 10%, 20%, 30%, 40%, 50%, 60%, 70%, 80%, 90%, 100% of the force at first peak tolerated by normal valves, i.e., 0.8 N. Alternatively, the recellularized valves withstand the force at first peak above 0.8 N.

As described herein, perfusion of peripheral whole blood results in the formation of a clear endothelial cell monolayer and presence of smooth muscle cells in the media of recellularized valve-bearing veins. Function and strength is preserved in the recellularized vein valves as determined by using the in vitro competency and reflux pressure tolerance test model and the biomechanical tear test. The use of a simple peripheral blood sample to recellularize the vein segments is a clear advantage over the more tedious approaches such as isolation and expansion of mature cells/stem cells from bone marrow or peripheral blood. These results are further supported by the successful transplantation of tissue-engineered veins using autologous peripheral whole blood to two paediatric patients.

### Methods of Treatment or Use

Tissue-engineered valve containing vein segments may be a therapeutic option in selected patients in whom deep venous reflux and venous hypertension are the main pathophysiological features leading to recurrent leg ulcerations. The present disclosure includes methods of treating and/or palliating venous disorders, e.g., deep vein thrombosis (DVT), chronic venous insufficiency (CVI) (also known as postphlebitic syndrome), and/or varicose veins by introducing or grafting recellularized valves and recellularized valve-bearing veins of the present disclosure to a subject in need.

The present disclosure provides a method for decellularizing and recellularizing a donor valve-bearing vein for treatment of chronic venous insufficiency and leg ulcerations. The recellularized valves produced by the methods described herein can be implanted, transplanted, or grafted into a patient suffering from or at risk of suffering from CVI and/or venous leg ulcerations.

The present disclosure further provides a method of treating or ameliorating symptoms of chronic venous insufficiency (CVI) and/or leg ulceration in a subject in need thereof, involving grafting recellularized valve-bearing segments of veins to the subject. The recellularization method includes one or more steps. The symptoms that are treated, ameliorated, or palliated may include: dull aching, heaviness, or cramping in legs, itching and tingling, pain that gets worse when standing, pain that gets better when legs are raised, swelling of the legs, redness of the legs and ankles, skin color changes around the ankles, varicose veins on the surface (superficial), thickening and hardening of the skin on the legs and ankles (lipodermatosclerosis), ulcers on the legs and ankles, and wound that is slow to heal on the legs or ankles.

The method includes harvesting valve-bearing segments of human femoral veins; decellularizing the veins in the femoral veins in 2-16 cycles; collecting blood from the subject; perfusing the decellularized veins with an anticoagulant; perfusing the decellularized veins with the collected blood for several hours; draining the blood and rinsing the vein with a buffer; and perfusing the veins first with endothelial cell media for more than one day, and then with smooth muscle media for more than one day; and thereby recellularizing the decellularized valves; where the recellularized valve upon grafting to the subject treats or ameliorates CVI and/or leg ulceration.

The present disclosure includes methods of treating recurrent leg cancer, which is due to deep venous reflux and/or venous hypertension, with recellularized valve-bearing veins. The present disclosure includes use of recellularized valve-bearing veins in treating recurrent leg cancer, which is due to deep venous reflux and/or venous hypertension.

The present disclosure includes recellularization of valves, which upon grafting in a subject in need thereof, treats and/or ameliorates the symptoms of incompetent valves in the thigh of the subject. In embodiments, the treatment and/or amelioration of the symptoms may be achieved by restoring normal working relationship between muscle pumps and the venous valves. The muscular pumps of the lower limb include those of the foot, calf, and thigh. A normal calf pump has the largest capacitance and generates the highest pressures (200 mm of mercury during muscular contraction). The normal limb has a calf volume ranging from 1500 to 3000 cc, a venous volume of 100 to 150 cc, and ejects over 40% to 60% of the venous volume with a single contraction.

During contraction, the gastrocnemius and soleus muscles drive blood into the large capacity popliteal and femoral veins. The recellularized valves of the present disclosure prevent retrograde flow (reflux) during subsequent relaxation, generating negative pressure and drawing blood from the superficial to the deep system through competent perforating veins. The recellularized valves incrementally lower venous pressure until arterial inflow equals venous outflow. The present disclosure includes that when exercise ceases in a subject, the veins with recellularized valves slowly fill the capillary bed, causing a slow return to the resting venous pressure.

Although muscle surrounds the thigh veins, the contribution of thigh muscle contraction to venous return is minimal compared with the calf muscle pump. Pumping action due to compression of the planter venous plexus during ambulation primes the calf pump. Various leg pumps work together with competent valve function to return venous blood from the distal to proximal extremity. The recellularized valves of the present disclosure are for use in restore functional leg pumps to return venous blood from the distal to proximal extremity.

The present disclosure includes a method for decellularizing and recellularizing a donor valve-bearing vein for treatment of chronic venous insufficiency and/or leg ulcerations. The recellularized valves produced by the methods described herein can be implanted, transplanted, or grafted into a patient suffering from or at risk of suffering from CVI and/or venous leg ulcerations.

Chronic venous insufficiency defines those manifestations of venous disease resulting from ambulatory venous hypertension, defined as a failure to reduce venous pressure with exercise. Under normal circumstances, the venous valves and the muscular pumps of the lower extremity limit the accumulation of blood in the lower extremity veins. Failure of the lower extremity muscle pumps due to out-flow obstruction, musculo-fascial weakness, loss of joint motion, or valvular failure is associated with peripheral venous insufficiency.

Venous ulcerations are wounds due to improper functioning of the venous valves, usually of the legs (i.e., venous leg uclerations). Venous ulcers arise when valves have reduced function and the backflow of blood causes pooling of blood in the veins and increased pressure in the veins and capillaries. This leads to other related complications, such as edema, inflammation, hardening of the tissue, malnutrition of the skin, and venous eczema. Venous ulcers are large, shallow, discolored due to leakage of iron-containing pigment in red blood cells into the tissue, and may have discharge. The ulcers are most frequently situated around the medial or later malleoli.

The present disclosure provides use of recellularized valves in veins for treating or ameliorating symptoms of venous diseases or disorder. For example, the present disclosure provides use of recellularized vein bearing valves in a method of treating or ameliorating chronic venous insufficiency or leg ulcers. The recellularized valves in veins of the present disclosure restore normal lower extremity venous pressure. For example, with walking, lower extremity venous pressure is reduced from approximately 100 mm Hg (depending on height) to mean of 18 mm Hg to about 25 mm Hg within 7 to 12 steps. Similar pressure changes are observed with standing ankle planter flexion or heel raising, transferring weight to the forefoot (the tiptoe maneuver). Ambulatory venous pressure (AVP) can be determined using a 21-gauge needle to measure the response to 10 tiptoe movements, usually at a rate of 1 per second, in dorsal foot vein. When resuming a stating standing position, hydrostatic pressure is restored after a mean of 31 seconds. The incidence of ulceration has a linear relationship to increases in AVP above 30 mm Hg. An increased AVP is also associated with a 90% venous refill time of less than 20 seconds. In contrast to the AVP, volume changes can be measure non-invasively using plethysmography. Rapid reflux (i.e., venous filling of greater than 7 ml/sec) and calf pump dysfunction are associated with a high incidence of ulceration. The recellularized valves in veins, upon grafting, restore the normal AVP, normal rapid reflux and/or normal calf pump dysfunction. Preferably, the recellularized valves in veins, upon grafting, restore the normal tolerance of reflux pressure of about 100 mm Hg, and reduced to a mean of about 18 mm Hg to about 25 mm Hg during walking 7 to 12 steps.

The following examples are illustrative, but not limiting, of the methods and compositions of the present disclosure. Other features and advantages of the present disclosure are apparent from the different examples. The provided examples illustrate different components and methodology useful in practicing the present disclosure. The examples do not limit the claimed subject matter. Based on the present disclosure the skilled artisan can identify and employ other components and methodology useful for practicing the present disclosure.

### EXAMPLES

### Harvesting of Valve-Bearing Veins

Vein segments including the common femoral vein, profunda femoral vein and femoral vein were harvested from adult human cadavers by using vascular surgical technique and carefully ligating all side branches. Valves were identified and 12 segments were cut with a margin of 3-4 cm on each end of the valves. The vein segments were thoroughly rinsed in phosphate buffered saline (PBS) containing 0.5% penicillin, 0.5% streptomycin, 0.5% amphotericin B and preserved at 4° C. The samples were transported within one week on ice to the laboratory.

### Decellularization and Characterization

Decellularization was carried out using 1% Triton, 1% Tri-n-butyl phosphate (TNBP) and 4 mg/L DNAse. The decellularized vein segments were evaluated for residual DNA content, staining with hematoxylin and eosin (HE) and Masson's trichrome (MT) using standard procedures for acellularity and quantification of various ECM proteins. Immunohistochemistry for detection of HLA class I and II antigens was performed by standard procedure.

DNA Quantification

DNA was extracted from normal and DC veins by using a commercially available kit. Twenty mgs of 7 normal and 9 decellularized vein samples were collected from different veins before and after decellularization and DNA extraction was carried out according to DNeasy® Blood & Tissue Handbook, included in the kit (69506, Qiagen, Sweden). Extracted DNA was quantified at 260 nm wavelength using a nanodrop. (ND-1000, Saveen Werner, USA).

### ECM Staining and Quantification

Briefly, to demonstrate collagen and the connective tissues, the formalin fixed vein tissue sections were re-fixed overnight at room temperature in Bouin's fixative, followed by using the Masson's Trichrome staining kit (cat No. 25088-1, Polysciences Inc., USA). The dyes employed during the staining procedure stained the collagen fibers blue, nuclei black and the cytoplasm and muscle fibers red.

### Acid-/Pepsin-Soluble Collagen Quantification

The acid-/pepsin-soluble collagen content in the decellularized ECM was measured using a Sircol soluble collagen assay kit (Biocolor). To extract acid-/pepsin-soluble collagen, the ECM specimens were digested with 0.5 M acetic acid containing 1% (w/v) pepsin (P7012; Sigma) for 48 h at 4° C. The soluble collagen was incubated with 1 mL Sircol dye reagent for 30 min at room temperature. The collagen-dye complex was precipitated by centrifugation at 10,000 g for 10 min, and the supernatant was removed. The pellets were dissolved in 1 mL alkali reagent, and the relative absorbance was measured in a 96-well plate at 555 nm using a microplate reader (PowerWave XS, Bio-Tek Instruments).

### Sulfated GAG Quantification

The sulfated GAG content in the decellularized ECM was measured using a Blyscan sulfated GAG assay kit (Biocolor). To extract sulfated GAG, the ECMs were digested with a 0.1 M phosphate buffer (pH 6.8) containing 125 μg/mL papain (Sigma), 10 mM cysteine hydrochloride (Sigma), and 2 mM EDTA (Sigma) for 4-6 h (until tissue is completely dissolved) at 65° C. The suspension was centrifuged at 10,000 g for 10 min. The extracted sulfated GAGs (100 ul) was mixed with 1 mL Blyscan dye and shaken for 30 min. The precipitate was collected by centrifugation for 10 min and then dissolved in 0.5 mL dissociation reagent. The absorbance was measured in a 96-well plate at 656 nm using a microplate reader.

### Soluble Elastin Quantification

The soluble elastin content in the decellularized ECM was measured using a Fastin elastin assay kit (Biocolor). To extract soluble elastin, the ECM was hydrolyzed with 0.25 M oxalic acid (Sigma) at 100° C. for 4-5 h (until tissue is completely dissolved). The insoluble residues were separated by centrifugation. The supernatant was collected, and the sediment underwent an additional extraction under the same conditions. The extracted soluble elastin was mixed with 1 mL Fastin dye and shaken for 90 min. The precipitate was collected by centrifugation for 10 min and then dissolved in 250 μL dissociation reagent. The absorbance was measured in a 96-well plate at 513 nm using a microplate reader.

### Sterility Control Test

Sterility during recellularization was evaluated by collecting one ml of perfused endothelial and smooth muscle media was collected after every two days during culture and tested for microbial contaminants. About 500 μls collected media were added to fluid Thioglycollate broth and plated on Tryptone soya agar plates and incubated at 37° C. for 14 days. Media exposed to outside air was used as positive control and only media only was used as negative control. The growth of fungi, aerobic and anaerobic bacterial were visualized and also measured in spectrophotometer for absorbance at 600 nm. Differences in absorbance were noted.

### Recellularization of Veins

On the day of recellularization, 20-25 ml peripheral venous blood was collected from healthy donors (age group 25-35 years) in sterile heparin coated vacutainer tubes and transported to the laboratory as soon as possible (within 2 hours). The volume of blood required was determined by the length of the vein and of the pipes used in the bioreactor.

The entire recellularization process was performed under sterile conditions and all perfusions were carried out in an incubator at 37° C. supplied with 5% CO₂. Before recellularization, the veins were perfused with heparin (Leopharma, Sweden) at a concentration of 50 IU/ml PBS for 2 h. The heparin was drained off and whole blood was immediately perfused for 48 h at 2 ml/min speed. The blood was then drained off and the vein was rinsed with PBS containing 1% penicillin-streptomycin-amphotericin until blood was completely removed. The vein was subsequently perfused four days with endothelial and four days with smooth muscle media. The complete endothelial medium was prepared using MCDB131 (Life technologies, Sweden) basal medium supplemented with 10% heat inactivated human AB serum (Life technologies, Sweden), 1% glutamine (Lonza, Denmark), 1% penicillin-streptomycin-amphotericin, and EGM2 single quote kit (Lonza, Denmark) that contained ascorbic acid, hydrocortisone, transferrin, insulin, recombinant human VEGF, human fibroblast growth factor, human epithelial growth factor, heparin and gentamycin sulfate. The complete smooth muscle medium was prepared using 500 ml Medium 231 (Life technologies, Sweden) supplied with 10% heat inactivated human AB serum, 1% penicillin-streptomycin amphotericin and 20 ml smooth muscle growth supplement (SMGS) (Life Technologies, Sweden). Vein scaffolds were recellularized for a total of ten days (manuscript submitted).

### Characterization of Recellularized Veins

To visualize the presence of endothelial cells, antibodies to CD31 (1:200) (Abcam, Germany) and vWF (1:100) (Santa Cruz, Germany) were selected and stained by Immunohistochemistry and immunofluorescence, while smooth muscle actin (1:50) (Abcam, Germany) was stained by immunohistochemistry to visualize smooth muscle cells.

### Biomechanical Analysis

The mechanical properties of the vein valve were evaluated by tearing the valve in the horizontal direction with the help of suture, 4-0 non-absorbable monofilament suture made of polypropylene. The veins were cut open with surgical scissors and the suture was placed and attached to the grips of an Instron 5566 (Instron, Norwood USA) (FIG. S1). A pre-load of 0.1 N and a test speed of 20 mm/minute was used, this made the valve fracture horizontally. The accuracy of the tensile tester is 0.5% in force and 0.5% in elongation, based on calibrations performed regularly according to ISO 7500-1:2004 and ISO 9513:1999. Force at first peak was measured and median force was calculated for each sample. In total six recellularised veins (n=12 valves) and four normal veins (n=8 valves) were tested.

### Functional In Vitro Testing of Valve-Bearing Veins

A custom-made test set-up was used to assess the functionality of the veins, before and after re-cellularization (FIG. 1). The vein was mounted vertically in the in vitro flow circuit and perfused with room-temperature saline. The vein was connected to the fluid circuit by conic connectors, secured with sutures at both ends. To mimic the flow through a vein in the deep venous system under the stress that occurs during walking and respiration, a commercial peristaltic pump (Baxter, Model no. 700044, Healthcare Corp., USA) delivered intermittent flow to the circuit.

A mechanical valve was used to regulate the flow direction through the circuit during outflow from the pump, and when the same valve was switched to open position, it achieved back flow in the vein until the valve closed. Reflux pressure in the vein was adjusted by the height of the column of fluid above the valve. Ultrasound Doppler technique was used to detect potential reflux at the site of the venous valve (9 MHz linear probe, Vivid E9, GE). To optimize the visualization with ultrasound, the vein segment was submerged in a plastic container filled with saline. A contrast agent (SonoVue, Bracco™) was administered in the saline solution to enhance the echogenicity and enable recordings of the flow and flow direction in the circuit, through the vein valve. The time from flow reversal until cessation of flow was used as a measure of valve closure time. In addition the vein diameter was measured at the site of the leaflet at a reflux pressure at 100 mmHg.

### Statistics

Results are presented as median and range. Mann-Whitney U tests were performed to compare effects of decellularization and recellularization on the valves. Paired two-tailed student T test was used for quantification of ECM and DNA. P<0.05 was considered to be a significant difference.

1.-8. (canceled)

9. A method of treating chronic venous insufficiency (CVI), deep vein thrombosis (DVT), and/or leg ulceration in a subject in need thereof, comprising introducing a recellularized valve-bearing segment of a vein to the subject, wherein the valve is recellularized by a method comprising:
a) decellularizing a valve-bearing segment of a vein, wherein the vein is allogeneic to the subject;
b) collecting blood from the subject, wherein the blood comprises progenitor cells for endothelial cells and progenitor cells for smooth muscle cells;
c) perfusing the decellularized valve-bearing segment with the collected blood;
d) culturing the cells in the lumen of the decellularized valve-bearing segment, thereby recellularizing the decellularized valve of the segment; and
e) grafting the recellularized valve-bearing segment to the subject; wherein the grafting treats CVI, DVT and/or leg ulceration in the subject.

10. The method of claim 9, wherein the leg ulcer is recurrent and is due to deep venous reflux and/or venous hypertension.

11. The method of claim 10, wherein the blood is peripheral venous blood or whole blood.

12. The method of claim 11, wherein the peripheral venous blood or the whole blood is introduced to the decellularized segment by injection or perfusion.

13. The method of claim 12, further comprising culturing the cells by perfusion of endothelial cell medium and smooth muscle cell medium.

14. The method of claim 13, wherein the perfusion of the endothelial cell medium and the smooth muscle cell medium are in alternation.

15. The method of claim 9, wherein the recellularized segment is CD31 positive, vWF positive, smooth muscle actin positive, and has nuclei.

16. The method of claim 9, wherein the recellularized segment has mechanical properties of withstanding force at first peak at or above 0.8 N.

17. The method of claim 9, wherein the recellularized segment has a closure time of equal to or less than 0.5 seconds.

18.-26. (canceled)
